(19) United States
(12) Patent Application Publication
(10) Pub. No.: US 2018/0258377 A1
(43) Pub. Date: Sep. 13, 2018

(54) FLOW PASSAGE MODULE AND CELL CULTURE APPARATUS USING SAME

(71) Applicant: Hitachi High-Technologies Corporation, Tokyo (JP)

(72) Inventors: Akihiro SHIMASE, Tokyo (JP); Toshinari SAKURAI, Tokyo (JP)

(21) Appl. No.: 15/761,114

(22) PCT Filed: Sep. 28, 2015
(86) PCT No.: PCT/JP2015/077236

§ 371 Date: Mar. 19, 2018

(51) Int. Cl. C12M 1/00; F16K 7/06; B81B 3/00; B01F 3/08; F04B 45/06

(52) U.S. Cl. CPC C12M 1/007; F16K 7/06; F04B 45/06; B01F 3/08; B81B 3/0097

(57) ABSTRACT

Provided are a flow passage module which can achieve complete liquid substitution in a circulating flow passage with a simple structure, and a cell culture apparatus using said flow passage module. A flow passage module comprises: a flexible branching section which is provided with a first branching flow passage connected with an inflow passage for a fluid, a second branching flow passage connected with an outflow passage, a third branching flow passage connected with an entry-side end part of a circulating flow passage, and a fourth branching flow passage connected with an exit-side end part of the circulating flow passage, and which enables the branching flow passages to be communicated with each other; and a communication state switching part which has opening/closing members for closing and opening the desired branching flow passage from among the plurality of branching flow passages.

### TECHNICAL FIELD

The present invention relates to a flow passage switching technique in an analyzing apparatus or a cell culture apparatus, and particularly relates to a flow passage module having a plurality of branching flow passages communicating with each other and capable of communicating with a desired branching flow passage, and a cell culture apparatus using the same.

### BACKGROUND ART

For example, PTL 1 is known as a method of performing culture while a culture solution is circulated in a circulation flow passage. PTL 1 discloses an apparatus for culturing microorganisms in a growth manner while a microbial suspension contained in a culture medium is circulated through the circulation flow passage. The culture apparatus has a suspension discharge port connected to the circulation flow passage, a culture medium supply tank connected to the circulation flow passage, and a test flow passage (sampling flow passage) branched from the circulation flow passage and through which a prescribed amount of the microbial suspension flows.

In addition, PTL 2 discloses a dialysis treatment apparatus, and discloses a configuration having an arterial blood circuit an arterial drip chamber which can be connected via a four way valve to a blood introduction port of a dialyzer having a plurality of hollow fiber membranes so as to purify blood. In a case where two passages are formed in the four way valve and a blood pump is normally rotated, the arterial blood circuit, the four way valve allows the arterial drip chamber, and the blood introduction port to be communicated in this order, thereby introduce patient's blood into the dialyzer. In addition, the configuration is disclosed as follows. In a case the blood pump is reversely rotated, the four way valve allows the blood introduction port, the arterial drip chamber, and the arterial blood circuit to be communicated in this order, thereby returning a dialysate the patient's artery. Furthermore, PTL 2 also discloses a configuration as follows. The four way valve is configured to include a bag formed from a flexible member and a clamp member. In response to normal rotation and reverse rotation of the blood pump, the dialysis treatment apparatus switches directions of the clamp member which clamps the bag.

### CITATION LIST

### Patent Literature

PTL 1: JP-A-2004-357575

PTL 2: JP-A-2005-87610

### SUMMARY OF INVENTION

### Technical Problem

However, according to PTL 1, a configuration is adopted as follows. Valves are respectively disposed in a flow passage connecting the suspension discharge port and the circulation flow passage to each other, a flow passage connecting the culture medium supply tank and the circulation flow passage to each other, and the test flow passage, and these different valves are operated. In this manner, the suspension is discharged, the culture medium is supplied to the circulation flow passage, and the suspension is circulated through the test flow passage. Therefore, it is not expected to reduce the number of components or to downsize the apparatus. Moreover, PTL 1 has no consideration with regard to how to fill the inside of the circulation flow passage with the microbial suspension contained in the culture medium (hereinafter, referred to as liquid substitution), and has a possibility that bubbles may be mixed into the microbial suspension.

In addition, according to the configuration disclosed in PTL 2, the two flow passages inside the four way valve are required to be accurately positioned by rotating the four way valve. In addition, in a case where the four way valve is configured to include the bag formed from the clamp member and the flexible member, an operation for changing the direction of the clamp member has to be necessarily performed, in addition to an opening/closing operation of the clamp member. Consequently, the liquid substitution is less likely to be realized.

Therefore, the present invention aims to provide a flow passage module which can realize complete liquid substitution of a circulation flow passage with a simple structure, and a cell culture apparatus using the same.

### Solution to Problem

In order to solve the above-described problem, a flow passage module according to the present invention includes a flexible branching section that includes a first branching flow passage connected to an end portion of an inflow passage of a fluid, a second branching flow passage connected to an end portion of an outflow passage, a third branching flow passage connected to an entry-side end portion of a circulation flow passage, and a fourth branching flow passage connected to an exit-side end portion of the circulation flow passage, and that enables the respective branching flow passages to communicate with each other, and a communication state switching part that has an opening/closing member which closes or opens a desired branching flow passage of the plurality of branching flow passages, and that moves the opening/closing member in one direction and presses and closes the desired branching flow passage so as to switch a first communication state where the third branching flow passage and the fourth branching flow passage communicate with each other and a second communication state where the first branching flow passage and the third branching flow passage communicate with each other and the second branching flow passage and the fourth branching flow passage communicate with each other.

In addition, a cell culture apparatus according to the present invention includes an inflow passage through which a cell suspension or a culture medium flows, a circulation flow passage through which the cell suspension or the culture medium is circulated, a pump that is installed in the circulation flow passage, a culture vessel that is installed on a downstream side of the pump and in the circulation flow passage, a collection bag that is connected to an outflow passage, and a flow passage module that is connected to the inflow passage, the circulation flow passage, and the outflow passage. The flow passage module has a flexible branching section that includes a first branching flow passage connected to an end portion of the inflow passage, a second branching flow passage connected to an end portion of the outflow passage, a third branching flow passage connected to an entry-side end portion of the circulation flow passage, and a fourth branching flow passage connected to an exit-side end portion of the circulation flow passage, and that enables the respective branching flow passages to communicate with each other, and a communication state switching part that has an opening/closing member which closes or opens a desired branching flow passage of the plurality of branching flow passages, and that moves the opening/closing member in one direction and presses and closes the desired branching flow passage so as to switch a first communication state where the third branching flow passage and the fourth branching flow passage communicate with each other and a second communication state where the first branching flow passage and the third branching flow passage communicate with each other and the second branching flow passage and the fourth branching flow passage communicate with each other.

### Advantageous Effects of Invention

According to the present invention, it is possible to provide a flow passage module which can realize complete liquid substitution of a circulation flow passage with a simple structure, and a cell culture apparatus using the same.

Objects, configurations, and advantageous effects other than those described above will be clarified from description of the following embodiments.

### BRIEF DESCRIPTION OF DRAWINGS

FIG. 1 is an overall schematic configuration diagram including a flow passage module according to an embodiment of the present invention.

FIG. 2 is an enlarged view of a branching member (flexible branching section) illustrated in FIG. 1.

FIG. 3 is a view for describing a method of forming the branching member (flexible branching section) illustrated in FIG. 1.

FIG. 4 is a schematic configuration diagram of a communication state switching part illustrated in FIG. 1.

FIG. 5 is a view illustrating an example of a shape and a mutual arrangement relationship of a pinch member (pressing member) and a support member which configure a communication state switching part.

FIG. 6 is a view illustrating another example of a shape and a mutual arrangement relationship of a pinch member (pressing member) and a support member which configure a communication state switching part.

FIG. 7 is an overall schematic configuration diagram including a flow passage module having the communication state switching part illustrated in FIG. 5.

FIG. 8 is an overall schematic configuration diagram including a flow passage module having the communication state switching part illustrated in FIG. 6.

FIG. 9 is a schematic configuration diagram of a flow passage module according to Embodiment 2 serving as another embodiment of the present invention, and is a view illustrating a second communication state.

FIG. 10 is a schematic configuration diagram of the flow passage module according to Embodiment 2, and is a view illustrating a first communication state.

FIG. 11 is a schematic configuration diagram of a flow passage module according to Embodiment 3 serving as another embodiment of the present invention, and is a view illustrating an operation for switching the second communication state to the first communication state.

FIG. 12 is a schematic configuration diagram of a flow passage module according to Embodiment 4 serving as another embodiment of the present invention, and is a view illustrating an operation for switching the second communication state to the first communication state.

FIG. 13 is a schematic configuration diagram of a flow passage module according to Embodiment 5 serving as another embodiment of the present invention.

FIG. 14 is an overall schematic configuration diagram of a cell culture apparatus having a flow passage module according to Embodiment 6 serving as another embodiment of the present invention.

FIG. 15 is a view illustrating a modification example of the cell culture apparatus illustrated in FIG. 14.

FIG. 16 is an overall schematic configuration diagram of a turbidity meter having a flow passage module according to Embodiment 7 serving as another embodiment of the present invention.

FIG. 17 is an overall schematic configuration diagram of a cell dispersing device having a flow passage module according to Embodiment 8 serving as another embodiment of the present invention.

FIG. 18 is an overall schematic configuration diagram of a cell number adjustment device having a flow passage module according to Embodiment 9 serving as another embodiment of the present invention.

### DESCRIPTION OF EMBODIMENTS

In the description herein, a branching member (to be described later) having a plurality of branching flow passages communicating with each other will be referred to as a flexible branching section, in some cases. In addition, a pinch member which moves in one direction will be referred to as a pressing member, in some case. A mechanism that brings desired branching flow passages inside the flexible branching section into a communication state in cooperation with the pressing member and a support member which maintains a stationary state will be referred to as a communication state switching part. In addition, the pinch member (pressing member) and the support member will be referred to as an opening/closing member, in some cases.

In addition, in the description herein, “liquid substitution” includes an operation for filling the inside of a circulation flow passage (to be described later) with a liquid, for example, such as a cell culture solution while preventing bubbles from being mixed therewith, or an operation for replacing different types of liquid with each other inside the circulation flow passage.

Hereinafter, embodiments according to the present invention will be described with reference to the drawings.

### Embodiment 1

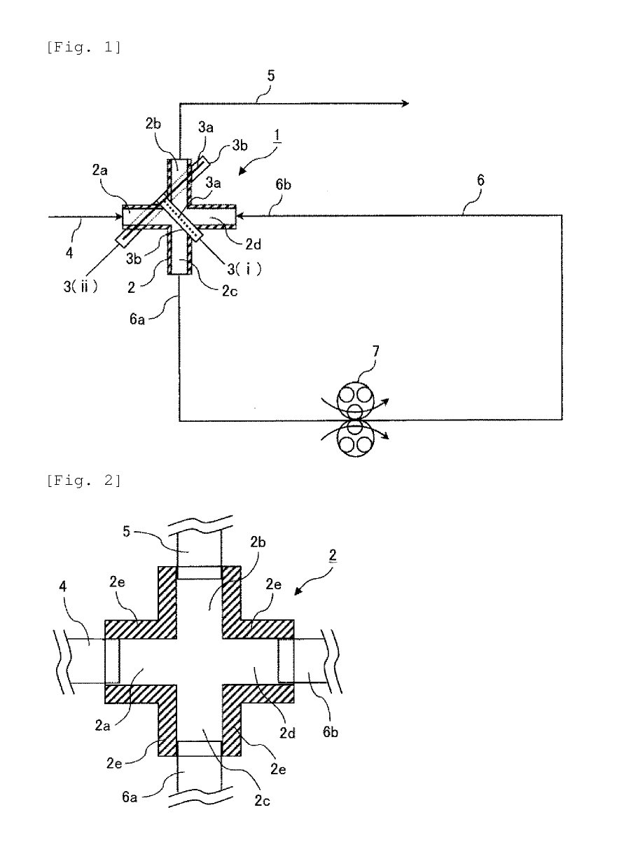

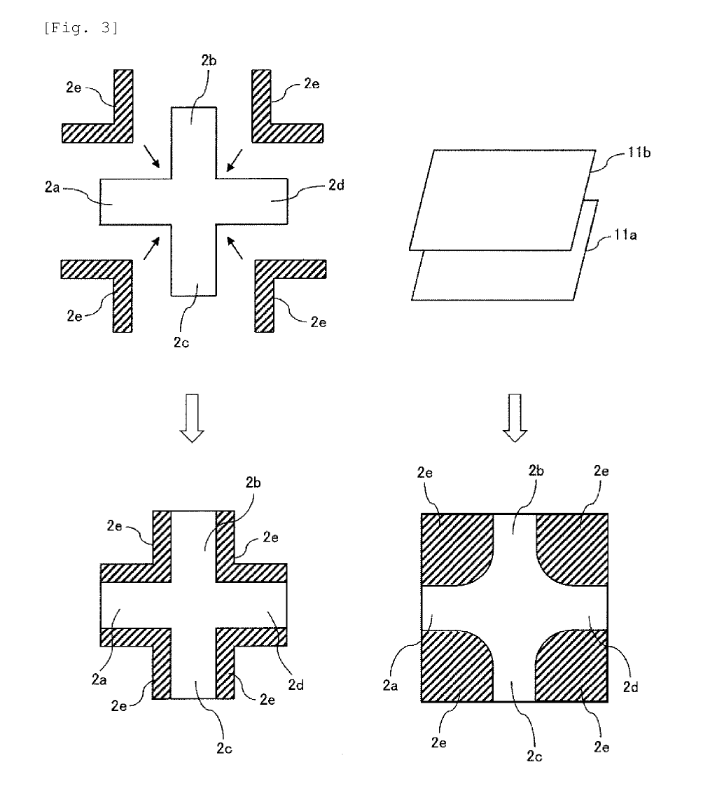

FIG. 1 is an overall schematic configuration diagram including a flow passage module according to an embodiment of the present invention. FIG. 2 is an enlarged view of a branching member (flexible branching section) illustrated in FIG. 1. As illustrated in FIG. 1, a flow passage module 1 according to the present embodiment includes a branching member (flexible branching section) 2 and a communication state switching part 3.

The branching member (flexible branching section) 2 includes four branching flow passages which communicate with each other, that is, a first branching flow passage 2a connected to an end portion of an inflow passage 4, a second branching flow passage 2b connected to an end portion of an outflow passage 5, a third branching flow passage 2c connected to entry-side end portion 6a of a circulation flow passage 6, and a fourth branching flow passage 2d connected to an exit-side end portion 6b of the circulation flow passage 6. In an example illustrated in FIG. 1, among the first branching flow passage 2a to the fourth branching flow passage 2d which configure the branching member 2, two adjacent branching flow passages are located so as to be orthogonal to each other in a horizontal plane. The first branching flow passage 2a connected to the inflow passage 4 and the fourth branching flow passage 2d connected to the exit-side end portion 6b of the circulation flow passage 6 face and communicate with each other. In addition, the second branching flow passage 2b connected to the outflow passage 5 and the third branching flow passage 2c connected to the entry-side end portion 6a of the circulation flow passage 6 face and communicate with each other.

The branching member (flexible branching section) 2 is formed from a flexible membrane (flexible sheet), and is located between a pinch member 3a and a support member 3b configuring the communication state switching part 3 (to be described later in detail). The branching member 2 is configured to be easily deformable by a pressing force of the pinch member 3a functioning as a pressing member. The communication state switching part 3 includes the pinch member (pressing member) 3a which moves in one direction and the support member 3b which maintains a stationary state. A set of the pinch member 3a and the support member 3b is located so as to interpose the first branching flow passage 2a and the second branching flow passage 2b therebetween. In addition, over a corner portion where the first branching flow passage 2a and the second branching flow passage 2b are joined to each other and a corner portion where the third branching flow passage 2c and the fourth branching flow passage 2d are joined to each other, another set of the pinch member 3a and the support member 3b is located in a planarly diagonal line shape passing through the center of the branching member 2.

The branching member 2 illustrated in FIG. 1 can be squeezed (pinched) in a direction of 3(i), that is, the branching member 2 can be squeezed by the set of the pinch member 3a and the support member 3b located in the planarly diagonal line shape. In addition, the branching member 2 can be squeezed (pinched) in a direction of 3 (ii), that is, the branching member 2 can be squeezed by the set of the pinch members 3a the support member 3b located so as to interpose the first branching flow passage 2a and the second branching flow passage 2b therebetween. If the branching member 2 is squeezed (pinched) in the direction of 3(i), the inflow passage 4 and the entry-side end portion 6a of the circulation flow passage 6 communicate (are continuous) with each other via the first branching flow passage 2a and the third branching flow passage 2c. The exit-side end portion 6b of the circulation flow passage 6 and the outflow passage 5 communicate (are continuous) with each other via the fourth branching flow passage 2d and the second branching flow passage 2b, thereby forming an open flow passage (open system) is formed and blocking the circulation flow passage. Hereinafter, this communication state will be referred to as a “second communication state”. On the other hand, if the branching member 2 is squeezed (pinched) in the direction of 3(ii), the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming a circulation flow passage (closed system) is formed and blocking the inflow passage 4 and the outflow passage 5. Hereinafter, this communication state will be referred to as a “first communication state”. In circulating a fluid (liquid), if the liquid does not flow to the branching member 2 from the outside and/or does not flow from the branching member 2 to the outside, the branching member 2 is not necessarily squeezed (pinched) in the direction of 3(ii). Therefore, from a viewpoint of preventing reverse flow, it is desirable to provide the set of the pinch member 3a and the support member 3b which squeeze (pinch) the branching member 2 in the direction of 3(ii) and which are located so as to interpose the first branching flow passage 2a and the second branching flow passage 2b therebetween. A pump 7 is disposed in the circulation flow passage 6. For example, as the pump 7, a squeezing pump for squeezing and feeding an elastic tube is used. In this case, for example, it is desirable that the circulation flow passage 6 is configured to include an elastic member such as a silicon tube.

(Configuration of Branching Member (Flexible Branching Section))

As illustrated in FIG. 2, the branching member (flexible branching section) 2 includes the first branching flow passage 2a, the second branching flow passage 2b, the third branching flow passage 2c, and the fourth branching flow passage 2d which are obtained by joining two flexible membranes (flexible sheets) to each other and which internally communicate with each other. An outer side portion of the respective branching flow passages are joined in a joint portion 2e, and a cross section of each branching flow passages is formed in a circular or elliptical shape. The cross section of each branching flow passage is not limited to the circular or elliptical shape, and may be a rectangular or polygonal shape. In this case, it is desirable to form a corner portion in a rounded shape. For example, as a joining method of the joint portion 2e, ultrasound welding or adhesion is used. Each branching flow passage may be joined to the branching member (flexible branching section) 2 in advance. That is, the joining may be performed by inserting the inflow passage 4 into the first branching flow passage 2a, inserting the outflow passage 5 into the second branching flow passage 2b, inserting the entry-side end portion 6a of the circulation flow passage 6 into the third branching flow passage 2c, and inserting the exit-side end portion 6b of the circulation flow passage 6 into the fourth branching flow passage 2d. FIG. 2 illustrates an example in which the inflow passage 4, the outflow passage 5, the exit-side end portion 6b of the circulation flow passage 6, and the entry-side end portion 6a of the circulation flow passage 6 are joined to each other in advance in the branching member (flexible branching section) 2. Alternatively, a configuration may be adopted in which the respective branching flow passages configuring the branching member 2 and the above-described inflow passage 4 are respectively connected via a connection component.

FIG. 3 is a view for describing a method of forming the branching member (flexible branching section) 2. As illustrated in the right drawing of FIG. 3, for example, a second flexible sheet 11b is aligned on a first flexible sheet 11a, and thereafter, the joint portions 2e are formed at four corners by means of ultrasound welding or thermal welding, for example, thereby forming the first branching flow passage 2a, the second branching flow passage 2b, the third branching flow passage 2c, and the fourth branching flow passage 2d, which are the four branching flow passages divided by the joint portions 2e. In addition, the configuration is not limited thereto. For example, as illustrated in the left drawing of FIG. 3, the joint portions 2e formed of different members may be joined so as to cover the outer side portions of the four branching flow passages which communicate with each other. In any case, since the configuration has the joint portions 2e, workability is improved when the branching member (flexible branching section) 2 is installed or mounted between the pinch member (pressing member) 3a and the support member 3b which configure the communication state switching part 3. It is not essential to adopt the configuration in which the joint portions 2e formed of different members are joined to the outer side portions of the four branching flow passages. A configuration may be adopted which uses the branching member (flexible branching section) 2 without having the joint portions 2e formed of the different members. In addition, it is not always necessary to form the branching member (flexible branching section) 2 by using the two sheets of the flexible sheets 11a and 11b, and one flexible sheet (flexible membrane) may be folded to form the branching member (flexible branching section) 2.

(Configuration of Communication State Switching Part)

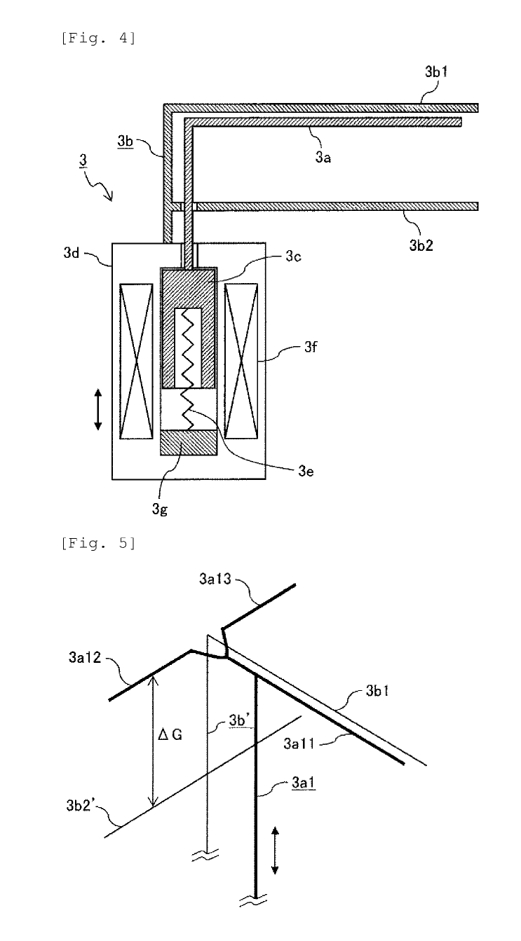

Next, a configuration of the communication state switching part 3 will be described. FIG. 4 is a schematic configuration diagram of the communication state switching part 3. As illustrated in FIG. 4, the communication state switching part 3 includes a pinch member (pressing member) 3a which moves in one direction, a support member 3b which maintains a stationary state, a movable iron core 3c fixed to one end portion of the pinch member 3a, a spring 3e in which one end is connected to a lower surface of the movable iron core 3c and the other end is connected to a fixed iron core 3g, a coil 3f, and a housing 3d which fixes one end portion of the support member 3b and which accommodates the movable iron core 3c, the spring 3e, and the coil 3f. The pinch member 3a which functions as a pressing member moves in one direction along with the movement of the movable iron core 3c. Hereinafter, the pinch member 3a and the movable iron core 3c are collectively referred to as an actuator. One end of the support member 3b is fixed to an upper surface of the housing 3d, thereby maintaining a stationary state at all times. The support member 3b is configured to include an erected portion erected upward in a vertical direction from an upper surface of the housing 3d, a normally closed side member (NC-side member) 3b1 which is bent in an upper end portion of the erected portion and which extends in a horizontal direction, and a normally open side member (NO-side member) 3b2 which is positioned below the NC-side member 3b1 at a predetermined interval and which extends in the horizontal direction from the erected portion. Here, the pinch member 3a and the support member 3b may be rigid. For example, both of these are formed of stainless steel, iron, or a resin. The branching member (flexible branching section) 2 is installed by being inserted between the support member 3b and the pinch member 3a. For convenience of description, FIG. 4 illustrates a simplified shape of the pinch member 3a and the support member 3b.

The actuator moves upward and downward in FIG. 4 by using a spring force generated by the spring 3e and a magnetic force generated by supplying power to the coil 3f. That is, the actuator moves in one direction. In a state where no power is supplied to the coil 3f, the actuator is actuated by the spring force of the spring 3e, and is pushed up toward the NC-side member 3b1 configuring the support member 3b. In this manner, the branching flow passage of the branching member (flexible branching section) 2 installed between the NC-side member 3b1 and the pinch member 3a is squeezed (pinched). On the other hand, if the power is supplied to the coil 3f, the actuator is attracted to the fixed iron core 3g side against the spring force of the spring e. In this manner, the branching flow passage of the branching member (flexible branching section) 2 installed between the NO-side member 3b2 and the pinch member 3a is squeezed (pinched). As a drive source of the actuator, in addition to a configuration utilizing an electromagnetic force as illustrated in this drawing, a configuration may be adopted which utilizes pressure such as air pressure or liquid pressure, or a mechanical force of a cam.

Alternatively, a rod may be located in place of the spring 3e illustrated in FIG. 4. The actuator may be configured to move upward and downward by switching between directions of a current flowing through the coil 3f.

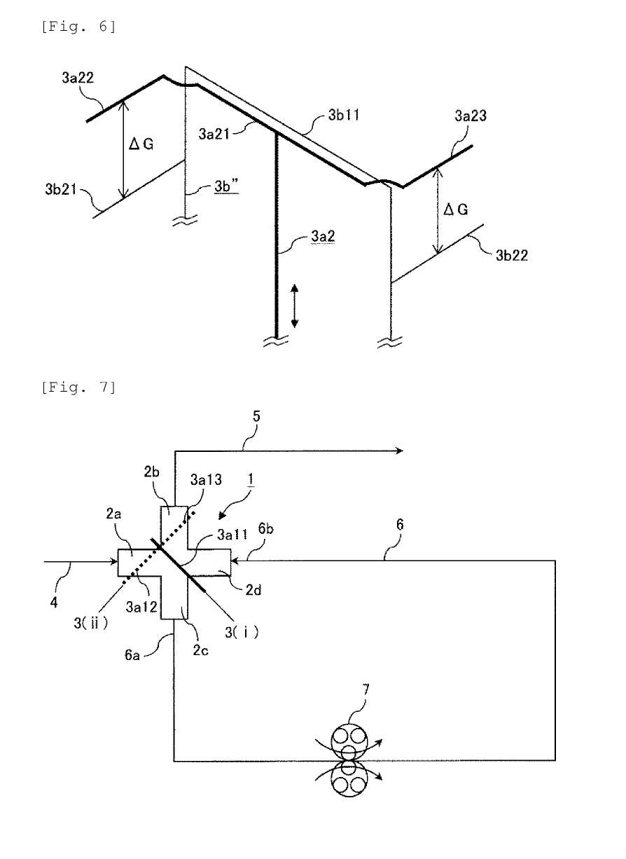

FIG. 5 is a view illustrating an example of a shape and a mutual arrangement relationship of the pinch member (pressing member) and the support member which configure the communication state switching part. In FIG. 5, a structure other than a pinch member (pressing member) 3a1 and a support member 3b′, that is, the coil 3f, the movable iron core 3c, the spring 3e, and the fixed iron core 3g which are illustrated in FIG. 4 described above are omitted in the illustration. The pinch member (pressing member) 3a1 and the support member 3b′1 configure the opening/closing member. A state illustrated in FIG. 5 indicates a case where no power is supplied to the coil 3f. As illustrated in FIG. 5, the support member 3b′ has an NC-side member 3b1 which is bent in the upper end portion of the erected portion and which extends in the horizontal direction, and an NO-side member 3b2′ which is positioned below the NC-side member 3b1 at a predetermined interval and which extends in the horizontal direction from the erected portion. The NC-side member 3b1 and the NO-side member 3b2′ are located so as to be orthogonal to each other in mutually different planes. In other words, the NC-side member 3b1 and the NO-side member 3b2′ are located so as to be orthogonal to each other in a vertical projection plane. In addition, the pinch member (pressing member) 3a1 includes an erected portion erected upward in the vertical direction, a first pressing portion 3a11 which is positioned in the upper end portion of the erected portion and which extends in the horizontal direction so as to face the NC-side member 3b1, a second pressing portion 3a12 which is located on one end side of the first pressing portion 3a11 and which extends in the horizontal direction so as to face the NO-side member 3b2′, and a third pressing portion 3a13 which is located on one end side of the first pressing portion 3a11 similarly to the second pressing portion 3a12 and which extends in the horizontal direction so as to face the NO-side member 3b2′. The second pressing portion 3a12 and the third pressing portion 3a13 are located so as to be orthogonal to the first pressing portion 3a11 in the same plane, and an end portion of the second pressing portion 3a12 on the first pressing portion 3a11 side and an end portion of the third pressing portion 3a13 on the first pressing portion 3a11 side are positioned so that respective extension lines are connected to each other. The end portion of the second pressing portion 3a12 on the first pressing portion 3a11 side and the end portion of the third pressing portion 3a13 on the first pressing portion 3a11 side are connected to each other in a V-shaped portion at a predetermined opening angle with the one side end portion of the first pressing portion 3a11. In this manner, the second pressing portion 3a12 and the third pressing portion 3a13 do not interfere with or come into contact with the erected portion of the support member 3b′.

In addition, as illustrated in FIG. 5, a gap ΔG between the second pressing portion 3a12 configuring the pinch member (pressing member) 3a1 and the NO-side member 3b2′ configuring the support member 3b′, and a gap ΔG between the third pressing portion 3a13 and the NO-side member 3b2′ may be equal to or larger than an outer diameter of the respective branching flow passages (2a, 2b, 2c, and 2d) disposed in the branching member (flexible branching section) 2. It is desirable that the gap ΔG is substantially equal to the outer diameter of the branching flow passages.

In a case where the gap ΔG illustrated in FIG. 5 is set to be substantially equal to the outer diameter of the branching flow passage disposed in the branching member (flexible branching section) 2, the branching member (flexible branching section) 2 is installed in the communication state switching part 3 as follows, for example. In a state where no power is supplied to the coil 3f illustrated in FIG. 4, the first branching flow passage 2a of the branching member (flexible branching section) 2 is inserted between the second pressing portion 3a12 configuring the pinch member (pressing member) 3a1 and the NO-side member 3b2′ configuring the support member 3b′. The second branching flow passage 2b is inserted between the third pressing portion 3a13 and the NO-side member 3b2′. Thereafter, the power is supplied to the coil 3f, the pinch member (pressing member) 3a1 is moved downward, and the first branching flow passage 2a is squeezed (pinched) by the second pressing portion 3a12 and the NO-side member 3b2. The second branching flow passage 2b is squeezed by the third pressing portion 3a13 and the NO-side member 3b2′. In this manner, the gap ΔG is formed between the NC-side member 3b1 configuring the support member 3b′ and the first pressing portion 3a11 configuring the pinch member (pressing member) 3a1. Here, the third branching flow passage 2c and the fourth branching flow passage 2d are inserted between the NC-side member 3b1 and the first pressing portion 3a11. The lower surface of the corner portion where the third branching flow passage 2c and the fourth branching flow passage 2d are joined to each other is positioned on the first pressing portion 3a11, and the upper surface of the corner portion where the third branching flow passage 2c and the fourth branching flow passage 2d are joined to each other is positioned directly below the NC-side member 3b1. Similarly, the lower surface of the corner portion where the first branching flow passage 2a and the second branching flow passage 2b are joined to each other is positioned on the first pressing portion 3a11, and the upper surface of the corner portion where the first branching flow passage 2a and the second branching flow passage 2b are joined to each other is positioned directly below the NC-side member 3b1. According to the above-described arrangement, the branching member (flexible branching section) 2 is completely installed in the communication state switching part 3.

The branching member (flexible branching section) 2 has three pinching positions in two directions. That is, in two directions of an extending direction of the first pressing portion 3a11 configuring the pinch member (pressing member) 3a1 and an extending direction of the second pressing portion 3a12 and the third pressing portion 3a13, the branching member 2 is pinched at three positions such as the first branching flow passage 2a pinched by the second pressing portion 3a12 and the NO-side member 3b2′, the second branching flow passage 2b pinched by the third pressing portion 3a13 and the NO-side member 3b2′, and the planarly diagonal line shape (over the corner portion where the first branching flow passage 2a and the second branching flow passage 2b are joined to each other and the corner portion where the third branching flow passage 2c and the fourth branching flow passage 2d are joined to each other) pinched by the first pressing portion 3a11 and the NC-side member 3b1.

FIG. 6 illustrates another example of a shape and a mutual arrangement relationship of a pinch member (pressing member) and a support member which configure a communication state switching part. Similarly to FIG. 5, in FIG. 6, a structure other than a pinch member (pressing member) 3a2 and a support member 3b″, that is, the coil 3f, the movable iron core 3c, the spring 3e, and the fixed iron core 3g which are illustrated in FIG. 4 described above are also omitted in the illustration. As illustrated in FIG. 6, the support member 3b″ has an NC-side member 3b11 which couples upper end portions of two erected portions erected in the vertical direction, a first NO-side member 3b21 which is positioned below the NC-side member 3b11 at a predetermined interval, whose one end is coupled to one erected portion, and which extends in the horizontal direction from the erected portion, and a second NO-side member 3b22 whose one end is coupled to the other erected portion, and which extends in the horizontal direction from the erected portion. The first NO-side member 3b21 and the second NO-side member 3b22 are positioned in the same horizontal plane, and the respective extending directions are mutually opposite directions. In addition, the first NO-side member 3b21 and the second NO-side member 3b22 are located so as to be orthogonal to the NC-side member 3b11 in a vertical projection plane. The pinch member (pressing member) 3a2 includes a first pressing portion 3a21 which extends in the horizontal direction around the upper end portion of the erected portion in the upper end portion of the erected portion erected in the vertical direction, a second pressing portion 3a22 which is located on one end side of the first pressing portion 3a21 and which extends in the horizontal direction so as to face the first NO-side member 3b21, and a third pressing portion 3a23 which is located on the other end side of the first pressing portion 3a21 and which extends in the horizontal direction so as to face the second NO-side member 3b22. An end portion of the second pressing portion 3a22 on the first pressing portion 3a21 side and an end portion of the first pressing portion 3a21 are connected to each other in an arc portion. In addition, an end portion of the third pressing portion 3a23 on the first pressing portion 3a21 side and an end portion of the first pressing portion 3a21 are connected to each other in an arc portion. In this manner, the erected portion of the support member 3b″ to which the first NO-side member 3b21 is connected and the second pressing portion 3a22 do not interfere with or come into contact with each other. Similarly, the erected portion of the support member 3b″ to which the second NO-side member 3b22 is connected and the third pressing portion 3a23 do not interfere with or come into contact with each other. As illustrated in FIG. 6, the second pressing portion 3a22 and the third pressing portion 3a23 extend in mutually opposite directions.

As illustrated in FIG. 6, the gap ΔG between the second pressing portion 3a22 configuring the pinch member (pressing member) 3a2 and the first NO-side member 3b21 configuring the support member 3b″ and the gap ΔG between the third pressing portion 3a23 and the second NO-side member 3b22 may be equal to or larger than the outer diameter of the respective branching flow passages (2a, 2b, 2c, and 2d) disposed in the branching member (flexible branching section) 2. It is desirable that the gap ΔG is substantially equal to the outer diameter of the branching flow passages.

In a case where the gap ΔG illustrated in FIG. 6 is set to be substantially equal to the outer diameter of the branching flow passage disposed in the branching member (flexible branching section) 2, the branching member (flexible branching section) 2 is installed in the communication state switching part 3. The installation will be described as follows. Here, a case is assumed where four branching flow passages disposed inside the branching member (flexible branching section) 2 are located so that the first branching flow passage 2a and the second branching flow passage 2b face and communicate with each other, and so that the third branching flow passage 2c and the fourth branching flow passage 2d face and communicate with each other. In a state where no power is supplied to the coil 3f illustrated in FIG. 4, the second branching flow passage 2b of the branching member (flexible branching section) 2 is inserted between the second pressing portion 3a22 configuring the pinch member (pressing member) 3a2 and the first NO-side member 3b21 configuring the support member 3b″, and the first branching flow passage 2a is inserted between the third pressing portion 3a23 and the second NO-side member 3b22. Thereafter, the power is supplied to the coil 3f, and the pinch member (pressing member) 3a2 is moved downward. The second branching flow passage 2b is squeezed by the second pressing portion 3a22 and the first NO-side member 3b21, and the first branching flow passage 2a is squeezed (pinched) by the third pressing portion 3a23 and the second NO-side member 3b22. In this manner, the gap ΔG is formed between the NC-side member 3b11 configuring the support member 3b″ and the first pressing portion 3a21 configuring the pinch member (pressing member) 3a2. Here, the third branching flow passage 2c and the fourth branching flow passage 2d are inserted between the NC-side member 3b11 and the first pressing portion 3a21. In this case, the lower surface of the corner portion where the third branching flow passage 2c and the fourth branching flow passage 2d are joined to each other is located on the first pressing portion 3a21, and the upper surface of the corner portion where the third branching flow passage 2c and the fourth branching flow passage 2d are joined to each other is located directly below the NC-side member 3b11. Similarly, the lower surface of the corner portion where the first branching flow passage 2a and the second branching flow passage 2b are joined to each other is located on the first pressing portion 3a21, and the upper surface of the corner portion where the first branching flow passage 2a and the second branching flow passage 2b are joined to each other is located directly below the NC-side member 3b11. According to the above-described arrangement, the branching member (flexible branching section) 2 is completely installed in the communication state switching part 3.

Compared to a configuration of the opening/closing member illustrated in FIG. 5, that is, a configuration of the pinch member (pressing member) 3a1 and the support member 3b′, in a configuration of the opening/closing member illustrated in FIG. 6, that is, a configuration of the pinch member 3a2 and the support member 3b″, a bending force applied to the pinch member is less generated. However, it does not particularly matter even when adopting either the configuration of the opening/closing member illustrated in FIG. 5 or the configuration of the opening/closing member illustrated in FIG. 6. Whether to adopt either the opening/closing member illustrated in FIG. 5 or the opening/closing member illustrated in FIG. 6 may be determined in view of the usage of the branching member and the accompanying component arrangement relationship. A shape may be used in which a positional relationship in a height direction between the NC-side member 3b1 and the NO-side member 3b2′ in FIG. 5 described above is reversed. However, in this case, it is necessary to change the shape so that the NC-side member 3b1 and the erected portion of the pinch member (pressing member) 3a1 do not interfere with or come into contact with each other. For example, a portion corresponding to the erected portion of the pinch member (pressing member) 3a1 of the NC-side member 3b1 is set to be an arc portion. Alternatively, a shape may be used in which the positional relationship in the height direction between the NC-side member 3b11 in FIG. 6, and the first NO-side member 3b21 and the second NO-side member 3b22 is reversed.

(Configuration and Operation of Flow Passage Module)

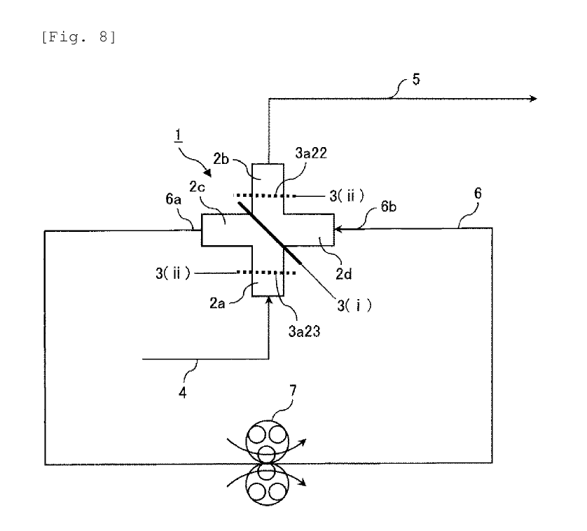

FIG. 7 is an overall schematic configuration diagram including a flow passage module having the communication state switching part illustrated in FIG. 5. As illustrated in FIG. 7, the flow passage module 1 includes the branching member (flexible branching section) 2 and the communication state switching part 3 illustrated in FIG. 5.

The branching member (flexible branching section) 2 includes the first branching flow passage 2a connected to the end portion of the inflow passage 4, the second branching flow passage 2b connected to the end portion of the outflow passage 5, the third branching flow passage 2c connected to the entry-side end portion 6a of the circulation flow passage 6, and the fourth branching flow passage 2d connected to the exit-side end portion 6b of the circulation flow passage 6. The first branching flow passage 2a and the fourth branching flow passage 2d face and communicate with each other, and the second branching flow passage 2b and the third branching flow passage 2c face and communicate with each other. The third branching flow passage 2c connected to the entry-side end portion 6a of the circulation flow passage 6 and the fourth branching flow passage 2d connected to the exit-side end portion 6b of the circulation flow passage 6 are located so as to be adjacent to each other.

First, the branching member (flexible branching section) 2 is squeezed (pinched) in the direction of 3(i) by supplying the power to the coil 3f configuring the communication state switching part 3 illustrated in FIG. 4. That is, in the first pressing portion 3a11 configuring the pinch member (pressing member) 3a1 and the NC-side member 3b1 (not illustrated in FIG. 7) configuring the support member 3b′ illustrated in FIG. 5, the branching member (flexible branching section) 2 is pinched into a planarly diagonal line to shape so as to cross the corner portion where the first branching flow passage 2a and the second branching flow passage 2b are joined to each other and the corner portion where the third branching flow passage 2c and the fourth branching flow passage 2d are joined to each other. In this manner, the inflow passage 4 and the entry-side end portion 6a of the circulation flow passage 6 communicate (are continuous) with each other via the first branching flow passage 2a and the third branching flow passage 2c. The exit-side end portion 6b of the circulation flow passage 6 and the outflow passage 5 communicate (are continuous) with each other via the fourth branching flow passage 2d and the second branching flow passage 2b, thereby forming the open flow passage (open system) and allowing the second communication state.

Next, the pump 7 installed in the circulation flow passage 6 is driven. Since the pump 7 is driven, the branching member (flexible branching section) 2 internally has negative pressure. Thus, for example, a liquid such as a culture solution is aspirated into the first branching flow passage 2a from the inflow passage 4. The liquid aspirated into the first branching flow passage 2a is introduced into the entry-side end portion 6a of the circulation flow passage 6 via the third branching flow passage 2c, and flows into the circulation flow passage 6. The liquid flows into the fourth branching flow passage 2d from the exit-side end portion 6b of the circulation flow passage 6. Thereafter, the liquid flows through the fourth branching flow passage 2d and the second branching flow passage 2b, and flows out to the outflow passage 5. The second communication state is continuously maintained for a predetermined period of time by the communication state switching part 3. In this manner, a gas phase (bubbles) present inside each of the branching flow passages (the first branching flow passage 2a to the fourth branching flow passage 2d) and inside the circulation flow passage 6 is discharged from the outflow passage 5. The liquid flows through the inner wall inside the first branching flow passage 2a to the fourth branching flow passage 2d, thereby discharging the bubbles adhering to the inner wall.

Thereafter, the power is supplied to the coil 3f illustrated in FIG. 4, thereby pinching the branching member (flexible branching section) 2 in the direction of 3 (ii). That is, the first branching flow passage 2a is pinched by the second pressing portion 3a12 configuring the pinch member (the pressing member) 3a1 and the NO-side member 3b2′ (not illustrated in FIG. 7) illustrated in FIG. 5, and the second branching flow passage 2b is pinched by the third pressing portion 3a13 and the NO-side member 3b2′. In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming the circulation flow passage (closed system) and allowing the first communication state.

In this way, the communication state switching part 3 allows the second communication state, drives the pump 7, and switches the second communication state to the first communication state after a predetermined period of time elapses. In this manner, it is possible to realize complete liquid substitution inside the circulation flow passage 6.

In addition, in a case where different liquids are replaced with each other, a first liquid flowing into the circulation flow passage 6 in the first communication state is switched to the second communication state by the communication state switching part 3, and is discharged from the outflow passage 5 via the fourth branching flow passage 2d and the second branching flow passage 2b of the branching member (flexible branching section) 2. Thereafter, for example, in a state where the second communication state is maintained, system water such as pure water is caused to flow through and discharged from the inflow passage 4, the first branching flow passage 2a, the third branching flow passage 2c, the circulation flow passage 6, the fourth branching flow passage 2d, the second branching flow passage, and the outflow passage 5 in this order. Thereafter, a second liquid different from the first liquid is caused to flow for a predetermined period of time in a state where the second communication state is maintained, and is discharged from the outflow passage 5. Thereafter, the second communication state is switched to the first communication state by the communication state switching part 3. In this manner, it is possible to realize the liquid substitution which is an operation for replacing different liquids with each other.

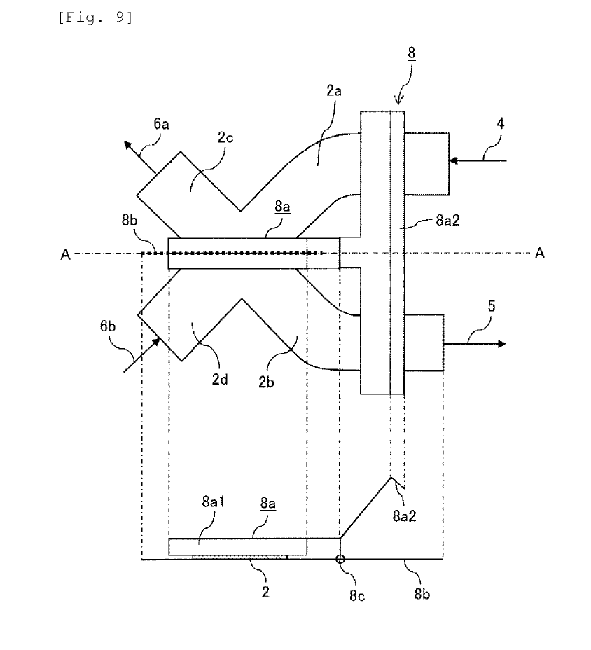

FIG. 8 is an overall schematic configuration diagram including the flow passage module having the communication state switching part illustrated in FIG. 6. As illustrated in FIG. 8, the flow passage module 1 includes the branching member (flexible branching section) 2 and the communication state switching part 3 illustrated in FIG. 6. To be strict, the pinching member (pressing member) 3a2 configuring the opening/closing member illustrated in FIG. 6 is provided with a shape in which the second pressing portion 3a22 and the third pressing portion 3a23 are not orthogonal to the first pressing portion 3a21 and are connected to the first pressing portion 3a21 at a predetermined angle (acute angle). In addition, similarly, the support member 3b″ configuring the opening/closing member is provided with a shape in which the first the NO-side member 3b21 and the second the NO-side member 3b22 are not orthogonal to the NC-side member 3b11 and are connected to the NC-side member 3b11 via the erected portion at a predetermined angle (acute angle). In addition, as illustrated in FIG. 8, in the branching member (flexible branching section) 2, the first branching flow passage 2a and the second branching flow passage 2b face and communicate with each other, and the third branching flow passage 2c and the fourth branching flow passage 2d face and communicate with each other. In other words, the entry-side end portion 6a of the flow passage 6 and the exit-side end portion 6b of the circulation flow passage 6 are located so as to face each other.

No power is supplied to the coil 3f configuring the communication state switching part 3 illustrated in FIG. 4, thereby squeezing (pinching) the branching member (flexible branching section) 2 in the direction of 3(i). That is, the first pressing portion 3a21 configuring the pinch member (pressing member) 3a2 and the supporting member 3b″ configuring the NC-side member 3b11 (not illustrated in FIG. 8) pinch the branching member (flexible branching section) 2 into a planarly diagonal line shape so as to cross the corner portion where the second branching flow passage 2b and the third branching flow passage 2c are joined to each other and the corner portion where the first branching flow passage 2a and the fourth branching flow passage 2d are joined to each other. In this manner, the inflow passage 4 and the entry-side end portion 6a of the circulation flow passage 6 communicate (are continuous) with each other via the first branching flow passage 2a and the third branching flow passage 2c, and the exit-side end portion 6b of the circulation flow passage 6 and the outflow passage 5 communicate (are continuous) with each other via the fourth branching flow passage 2d and the second branching flow passage 2b, thereby forming the open flow passage (open system) and allowing the second communication state.

In addition, the power is supplied to the coil 3f illustrated in FIG. 4, thereby pinching the branching member (flexible branching section) 2 in the direction of 3 (ii). That is, the second branching flow passage 2b is pinched by the second pressing portion 3a22 configuring the pinch member (pressing member) 3a2 and the first NO-side member 3b21 (not illustrated in FIG. 8). The first branching flow passage 2a is pinched by the third pressing portion 3a23 and the second NO-side member 3b22. In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming the circulation flow passage (closed system) and allowing the first communication state.

The operation for switching from the second communication state to the first communication state which is performed by the communication state switching part 3 at the time of the liquid substitution is the same as that in FIG. 7 described above, and thus, the description will be omitted.

Compared to the configuration of the flow passage module 1 illustrated in FIG. 7, the configuration of the flow passage module 1 illustrated in FIG. 8 has an advantageous effect in that the liquid transfer can be smoothly supplied in the circulation (first communication state). The reason is that the entry-side end portion 6a of the circulation flow passage 6 and the exit-side end portion 6b of the circulation flow passage 6 are located so as to face each other. On the other hand, similarly to the flow passage module 1 illustrated in FIG. 8, the configuration of the flow passage module 1 illustrated in FIG. 7 also has an advantageous effect in that a planar arrangement is available since the four branching flow passages (the first branching flow passage 2a to the fourth branching flow passage) do not intersect each other in the height direction. Therefore, whether to use either the flow passage module 1 illustrated in FIG. 7 or the flow passage module 1 illustrated in FIG. 8 may be determined depending on a demand needed by the apparatus. As illustrated in FIGS. 7 and 8, if the liquid supply direction at the time of the liquid substitution and the liquid supply direction at the time of the circulation are the same as each other, the pump 7 is more easily controlled. However, it is not always necessary to cause the liquid supply direction at the liquid substitution and the liquid supply direction at the time of the circulation to be the same as each other. The liquid supply direction may be changed depending on the use purpose.

As described above, according to the present embodiment, it is possible to realize the complete liquid substitution of the circulation flow passage with a simple structure.

### Embodiment 2

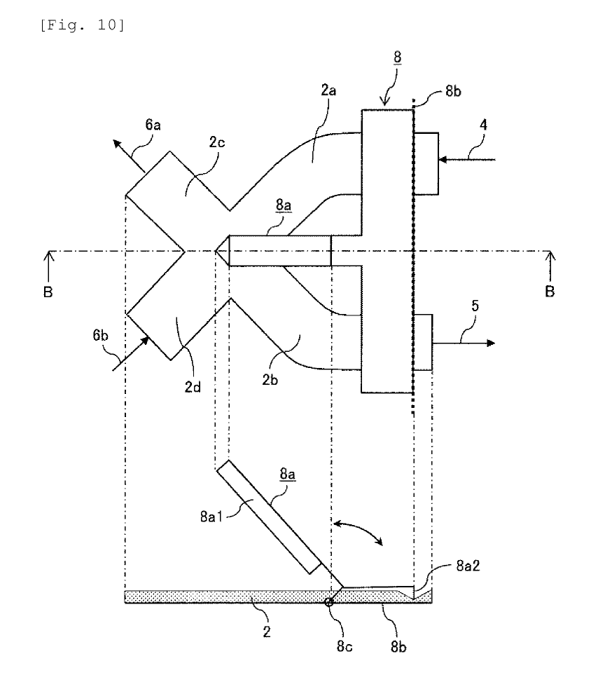

FIG. 9 is a schematic configuration diagram of the flow passage module according to Embodiment 2 serving as another embodiment of the present invention, and is a view illustrating the second communication state. FIG. 10 is a schematic configuration diagram of the flow passage module according to Embodiment 2, and illustrates the first communication state. In Embodiment 1, a configuration is adopted in which the actuator including the pinch member (pressing member) (3a, 3a1, and 3a2) and the movable iron core 3c which configure the communication state switching part 3 moves upward and downward in one direction (vertical direction) so as to squeeze (pinch) the branching member (flexible branching section) 2 in cooperation with the pin member and the support member which always maintains a stationary state, and so as to switch between the first communication state and the second communication state. In contrast, the present embodiment adopts a different configuration in that the pinch member (pressing member) pivots around a hinge (fulcrum) with respect to the support member so as to switch between the first communication state and the second communication state. The other configuration elements are the same as those in Embodiment 1. Hereinafter, repeated description in Embodiment 1 will be omitted. In FIGS. 9 and 10, the same reference numerals will be given to configuration elements which are the same as those in Embodiment 1.

FIG. 9 illustrates a top view of the flow passage module 1 in the upper drawing, and the lower drawing illustrates a sectional view taken along line A-A in the upper drawing. The flow passage module 1 includes the branching member (flexible branching section) 2 and a communication state switching part 8. As illustrated in the upper drawing and the lower drawing in FIG. 9, the communication state switching part 8 has an opening/closing member including a pinch member (pressing member) 8a and a support member 8b which always maintains a stationary state. The pinch member 8a includes a first pressing portion 8a1 having a T-shape in a plan view or in a vertical projection plane and extending in one direction, and a second pressing portion 8a2 extending in a direction perpendicular to the first pressing portion 8a1. In addition, the support member 8b is configured to include a portion having a cross shape in a plan view and extending in one direction while facing the first pressing portion 8a1 configuring the pinch member 8a, and a portion extending in the other direction while facing the second pressing portion 8a2 configuring the pinch member 8a.

As illustrated in the upper drawing in FIG. 9, in the pinch member 8a, one end of the first pressing portion 8a1 is connected to a substantially central portion of the second pressing portion 8a2. In addition, as illustrated in the lower drawing in FIG. 9, the second pressing portion 8a2 is provided with a cross-sectional shape which is inclined upward at a predetermined angle from the portion connected to the first pressing portion 8a1, which is bent at a predetermined position, and which is inclined downward at a predetermined angle. In addition, as illustrated in the lower drawing in FIG. 9, the pinch member 8a and the support member 8b are connected to each other via the hinge 8c disposed in the support member 8b. A location where the pinch member 8a is connected to the hinge 8c is a connection portion between the first pressing portion 8a1 and the second pressing portion 8a2. The pinch member 8a pivots in an arc shape around the hinge 8c serving as a fulcrum, that is, the pinch member 8a pivots in one direction which is a direction tracing an arc track. In this manner, the first pressing portion 8a1 and the second pressing portion 8a2 which configure the pinch member 8a pivot in an arc shape in mutually opposite directions. Here, a drive force for the pinch member 8a to pivot in the arc shape around the hinge 8c serving as the fulcrum is supplied, for example, by a motor (not illustrated) or a drive mechanism in which a spring and a linear motion mechanism such as an electromagnetic valve are combined with each other.

The pinch member 8a and the support member 8b may be rigid. For example, both of these are formed of stainless steel, iron, or a resin.

As illustrated in the upper drawing in FIG. 9, the branching member (the flexible branching section) 2 is located on the support member 8b facing the first pressing portion 8a1 in a planarly diagonal line shape across the corner portion where the third branching flow passage 2c connected to the entry-side end portion 6a of the circulation flow passage 6 and the fourth branching flow passage 2d connected to the exit-side end portion 6b of the circulation flow passage 6 are joined to each other and the corner portion where the first branching flow passage 2a connected to the inflow passage 4 and the second branching flow passage 2b connected to the outflow passage 5 are joined to each other. In addition, the first branching flow passage 2a and the second branching flow passage 2b are located on the support member 8b facing the second pressing portion 8a2. A flow passage length of the first branching flow passage 2a and the second branching flow passage 2b is longer than a flow passage length of the third branching flow passage 2c and the fourth branching flow passage 2d.

In a state illustrated in FIG. 9, the first pressing portion 8a1 of the pinch member 8a and the support member 8b which configure the opening/closing member brings the branching member (flexible branching section) 2 into a state pinched in a planarly diagonal line shape so as to cross the corner portion where the first branching flow passage 2a and the second branching flow passage 2b are joined to each other and the corner portion where the third branching flow passage 2c and the fourth branching flow passage 2d are joined to each other. In this manner, the inflow passage 4 and the entry-side end portion 6a of the circulation flow passage 6 communicate (are continuous) with each other via the first branching flow passage 2a and the third branching flow passage 2c, and the circulation flow passage 6 of the exit-side end portion 6b and the outflow passage 5 communicate (are continuous) with each other via the fourth branching flow passage 2d and the second branching flow passage 2b, thereby forming the open flow passage (open system) and allowing the second communication state.

FIG. 10 illustrates a state switched to the first communication state by the communication state switching part 8. FIG. 10 illustrates a top view of the flow passage module 1 in the upper drawing, and the lower drawing illustrates a sectional view taken along arrow B-B in the upper drawing. As illustrated in the lower drawing in FIG. 10, when the second communication state illustrated in FIG. 9 is switched to the first communication state, a drive mechanism in which a motor (not illustrated) or a spring and a linear motion mechanism such as an electromagnetic valve are combined with each other causes the pinch member (pressing member) 8a to pivot in an arc shape around the hinge 8c serving as a fulcrum. That is, in cooperation with the support member 8b so far, the first pressing portion 8a1 configuring the pinch member 8a pivots in the arc shape around the hinge 8c serving as the fulcrum, in a state where the branching member (flexible branching section) 2 is pinched in a planarly diagonal line shape so as to cross the corner portion where the first branching flow passage 2a and the second branching flow passage 2b are joined to each other and the corner portion where third branching flow passage 2c and the fourth branching flow passage 2d are joined to each other. In response to the pivoting of the first pressing portion 8a1, the second pressing portion 8a2 configuring the pinch member 8a pivots downward in an arc shape. Then, as illustrated in the upper drawing and the lower drawing in FIG. 10, in cooperation with the support member 8b, the second pressing portion 8a2 pinches the first branching flow passage 2a and the second branching flow passage 2b which configure the branching member (flexible branching section) 2 at the same time. In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming the circulation flow passage (closed system) and allowing the first communication state.

The lower drawing in FIG. 10 is a sectional view taken along an arrow B-B in the upper drawing. Accordingly, the lower drawing illustrates the branching member (flexible branching section) 2 located on the upper surface of the support member 8b throughout the longitudinal direction of the support member 8b which always maintains a stationary state. However, in actual, the branching member (flexible branching section) 2 is located on the support member 8b which extends while facing the first pressing portion 8a1 configuring the pinch member (pressing member) 2, in a range from the corner portion where the first branching flow passage 2a and the second branching flow passage 2b are joined to each other to the corner portion where the third branching flow passage 2c and the fourth branching flow passage 2d are joined to each other through the center of the branching member 2.

In the present embodiment, the support member 8b is formed in a cross shape in a plan view. However, the configuration is not necessarily limited thereto. For example, the support member 8b may be configured to include a portion extending in one direction while facing the first pressing portion 8a1 configuring the pinch member 8a and a portion extending in the other direction while facing the second pressing portion 8a2 configuring the pinch member 8a. As illustrated in the lower drawing in FIG. 10, the support member 8b may be disposed at a position facing the second pressing portion 8a2, and may be formed so as to have a T-shape in a plan view.

According to the present embodiment, without considering a case of avoiding interference or contact between the pinch member (pressing member) and the support member as in Embodiment 1, it is possible to form the opening/closing member including the pinch member and the support member. In this manner, compared to Embodiment 1, it is possible to realize the complete liquid substitution of the circulation flow passage with a simpler structure.

### Embodiment 3

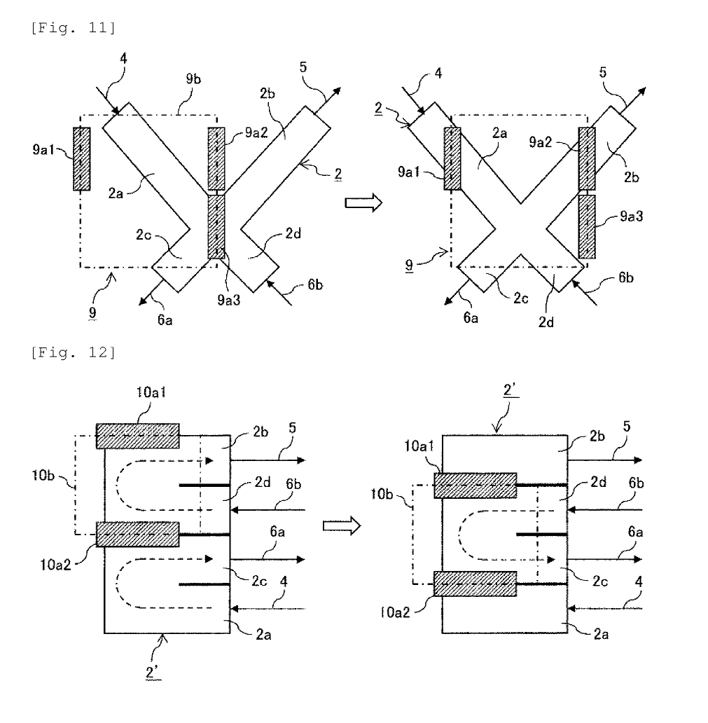

FIG. 11 is a schematic configuration diagram of the flow passage module according to Embodiment 3 serving as another embodiment of the present invention, and illustrates an operation for switching the second communication state to the first communication state. The present embodiment is different from Embodiment 1 and Embodiment 2 described above in that a plurality of rollers (pressing members) and a flat plate-shaped support member are located on a side opposite to the plurality of rollers across the branching member (flexible branching section) 2 so as to configure the opening/closing member. The other configuration elements are the same as those of Embodiment 1. Thus, hereinafter, repeated description in Embodiment 1 will be omitted. In FIG. 11, the same reference numerals will be given to configuration elements which are the same as those in Embodiment 1 and Embodiment 2 described above.

As illustrated in FIG. 11, the flow passage module 1 according to the present embodiment includes the branching member (flexible branching section) 2 and a communication state switching part 9. The branching member (flexible branching section) 2 includes the first branching flow passage 2a connected to the inflow passage 4, the second branching flow passage 2b connected to the outflow passage 5, the third branching flow passage 2c connected to the entry-side end portion 6a of the circulation flow passage 6 (not illustrated), and the fourth branching flow passage 2d connected to the exit-side end portion 6b of the circulation flow passage 6. The first branching flow passage 2a and the fourth branching flow passage 2d are located so as to face and communicate with each other, and the second branching flow passage 2b and the third branching flow passage 2c are located so as to face and communicate with each other. As illustrated in FIG. 11, the flow passage length of the first branching flow passage 2a and the second branching flow passage 2b is longer than the flow passage length of the third branching flow passage 2c and the fourth branching flow passage 2d.

The opening/closing member configuring the communication state switching part 9 is configured to include a flat plate-shaped support member (not illustrated) located in a depth direction from the branching member (flexible branching section) 2 in FIG. 11, and three rollers of a roller 9a1 (hereinafter, referred to as a first roller), a roller 9a2 (hereinafter, referred to as a second roller), and a roller 9a3 (hereinafter, referred to as a third roller) which are located above the branching member (flexible branching section) 2 and rotatably supported by a linear or rod-shaped connection member 9b having a quadrangular shape. The connection member 9b is configured to include a linear or rod-shaped portion having two sides which are parallel to a line segment (hereinafter, referred to as a diagonal line of the branching member 2) passing through the corner portion where the first branching flow passage 2a and the second branching flow passage 2b are joined to each other and the corner portion where the third branching flow passage 2c and the fourth branching flow passage are joined to each other and which face each other while being apart from each other at a predetermined interval, and a linear or rod-shaped portion having the other two sides which connect both end portions of the linear or rod-shaped portion having two sides and which face each other while being apart from each other at a predetermined interval. The first roller 9a1 is rotatably supported by one linear or rod-shaped portion located so as to be parallel to the diagonal line of the branching member 2 in the connection members 9b, and the second roller 9a2 and the third roller 9a3 are rotatably supported by the other linear or rod-shaped portion. In FIG. 11, the first roller 9a1 is located above the central portion in the longitudinal direction, which is the linear or rod-shaped portion on the left side of the two sides located so as to be parallel to the diagonal line of the branching member 2. In addition, the second roller 9a2 is located above the central portion in the longitudinal direction, and the third roller 9a2 is located below the central portion in the longitudinal direction. The second roller 9a2 and the third roller 9a3 are located while being apart from each other at a small interval along the longitudinal direction.

The first roller 9a1, the second roller 9a2, and the third roller 9a3 have substantially the same length in the longitudinal direction, and have the length which is equal to or longer than the line segment connecting the corner portion where the first branching flow passage 2a and the second branching flow passage 2b are joined to each other and the corner portion where the third branching flow passage 2c and the fourth branching flow passage 2d are joined to each other. In addition, the length of the three rollers 9a1 to 9a3 in the longitudinal direction is set to the length suitable for squeezing (closing) the first branching flow passage 2a and the second branching flow passage 2b (to be described later). In addition, an area of the plate-shaped support member (not illustrated) is at least larger than an area of the largest circumscribing rectangle of the branching member (flexible branching section) 2. In this manner, a configuration is adopted as follows. The branching member (flexible branching section) 2 can be placed on the plate-shaped support member. The first roller 9a1 to the third roller 9a3 and the connection member 9b which rotatably supports and connects these rollers are movable in one direction while pinching the branching member (flexible branching section) 2 with the above-described support member. A force or a load is always applied to the first roller 9a1 to the third roller 9a3 in a direction in which the branching member (flexible branching section) 2 is squeezed.

In a state illustrated in the left drawing in FIG. 11, in cooperation with the third roller 9a3 and the flat plate-shaped support member, the branching member (flexible branching section) 2 is brought into a state squeezed (closed) in a planarly diagonal line shape across the corner portion where the first branching flow passage 2a and the second branching flow passage 2b are joined to each other and the corner portion where third branching flow passage 2c and the fourth branching flow passage 2d are joined to each other. In this manner, the inflow passage 4 and the entry-side end portion 6a of the circulation flow passage 6 communicate (are continuous) with each other via the first branching flow passage 2a and the third branching flow passage 2c, and the exit-side end portion 6b of the circulation flow passage 6 and the outflow passage 5 communicate (are continuous) with each other via the fourth branching flow passage 2d and the second branching flow passage 2b, thereby forming the open flow passage (open system) and allowing the second communication state.

In a state illustrated in the left drawing in FIG. 11, the first roller 9a1 to the third roller 9a3 and the connection member 9b which rotatably supports and connects these rollers are moved in the horizontal direction (direction from the left side toward the right side in FIG. 11) by an actuator (not illustrated), for example. This state is illustrated in the right drawing in FIG. 11. As illustrated in the right drawing in FIG. 11, in cooperation with the first roller 9a1 and the flat plate-shaped support member, the first branching flow passage 2a is squeezed (closed), and at the same time, in cooperation with the second roller 9a2 and the flat plate-shaped support member, the second branching flow passage 2b is squeezed (closed). In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming the circulation flow passage (closed system) and allowing the first communication state.

In the present embodiment, a configuration is adopted in which the first roller 9a1 is located in the linear or rod-shaped portion on the left side configuring the connection member 9b and the second roller 9a2 and the third roller 9a3 are located in the linear or rod-shaped portion on the right side configuring the connection member 9b. However, the configuration is not limited thereto. For example, a configuration may be adopted in which the third roller 9a3 is located below the central portion in the longitudinal direction of the linear or rod-shaped portion on the left side, which is the linear or rod-shaped portion on the left side configuring the connection member 9b. In this case, the first roller 9a1 to the third roller 9a3 and the connection member 9b which rotatably supports these rollers are moved in the horizontal direction from the right side to the left side in FIG. 11 by an actuator (not illustrated). In this manner, the second communication state can be switched to the first communication state.

In addition, both end portions of the linear or rod-shaped portion having two sides configuring the connection member 9b which rotatably support the first roller 9a1 to the third roller 9a3 are connected to each other, and the linear or rod-shaped portion having the other two sides which face each other while being apart from each other at the predetermined interval is linearly formed. However, the configuration is not necessarily limited thereto. For example, the other two sides may be formed in an arc shape. Any shape may be employed as long as a structure is used which connects both end portions of the linear or rod-shaped portion having two sides rotatably supporting the first roller 9a1 to the third roller 9a3.

In addition, in the present embodiment, the first roller 9a1 to the third roller 9a3, the connection member 9b which rotatably supports the rollers, and the flat plate-shaped support member may be formed of a rigid material, for example, such as stainless steel, iron, or a resin.

According to the present embodiment, a configuration is adopted in which the first roller 9a1 to the third roller 9a3 are moved in the horizontal direction so as to switch the communication state of the branching member (flexible branching section), that is, a configuration in which the communication state is switched using a sliding method. Accordingly, compared to Embodiment 1 and Embodiment 2, it is expected that the switching time required for switching the second communication state to the first communication state is lengthened a little. However, in the present embodiment, it is possible to switch the communication state by sliding the rollers in one direction in the horizontal plane. Therefore, the apparatus can be further simplified.

### Embodiment 4

FIG. 12 is a schematic configuration diagram of the flow passage module according to Embodiment 4 serving as another embodiment of the present invention, and illustrates an operation for switching the second communication state to the first communication state. In FIG. 12, a flow of a liquid, for example, such as a culture solution flowing in the branching member (flexible branching section) is indicated by a dotted arrow. The present embodiment is different from Embodiment 3 in that all of the four branching flow passages such as the first branching flow passage 2a to the fourth branching flow passage 2d of the branching member (flexible branching section) are aligned in the same direction, and that the opening/closing member is configured to include two rollers (pressing members) and a flat plate-shaped support member (not illustrated) located on a side opposite to the two rollers across the branching member (flexible branching section). The other configuration elements are the same as those of Embodiment 1. Thus, hereinafter, repeated description in Embodiment 1 will be omitted.

As illustrated in FIG. 12, the flow passage module 1 according to the present embodiment includes a branching member (flexible branching section) 2′ and a communication state switching part 10. The branching member (flexible branching section) 2′ includes the first branching flow passage 2a connected to the inflow passage 4, the third branching flow passage 2c connected to the entry-side end portion 6a of the branching flow passage 6 (not illustrated), the fourth branching flow passage 2d connected to the exit-side end portion 6b of the branching flow passage 6, and the second branching flow passage 2b connected to the outflow passage 5. These four branching flow passages are adjacent to each other in the above-described order, and all of these are aligned in the same direction. In other words, the first branching flow passage 2a to the fourth branching flow passage 2d are located on the same side surface of the branching member (flexible branching section) 2′ having a quadrangular shape in a plan view. As illustrated in FIG. 12, in three flow passage walls extending in the horizontal direction from the right side surface to the central portion side of the branching member (flexible branching section) 2′, the first branching flow passage 2a, the third branching flow passage 2c, the fourth branching flow passage 2d, and the second branching flow passage 2b are respectively divided sequentially from the lower side. Then, these four branching flow passages can communicate with each other in a region from the left side end portion (central side end portion of the branching member) of the three flow passage walls to the left side surface of the branching member (flexible branching section) 2′. Here, for example, the three flow passage walls are formed in such a way that one flexible sheet is folded and subjected to ultrasound welding or heat welding.

The opening/closing member configuring the communication state switching part 10 is configured to include the flat plate-shaped support member (not illustrated) located in the depth direction from the branching member (flexible branching section) 2′ in FIG. 12 and two rollers of a roller 10a1 (hereinafter, referred to as a first roller) and a roller 10a2 (hereinafter, referred to as a second roller) which are located above the branching member (flexible branching section) 2′ and which are rotatably supported by the linear or rod-shaped connection member 10b having a quadrangular shape. The connection member 10b is configured to include a linear or rod-shaped portion having two sides which are parallel to the three flow passage walls and which face each other while being apart from each other at a predetermined interval, and a linear or rod-shaped portion having the other two sides which are orthogonal to the three flow passage walls and which face each other while being apart from each other at a predetermined interval. The first roller 10a1 is rotatably supported by one linear or rod-shaped portion located so as to be parallel to the three flow passage walls, and the second roller 10a2 is rotatably supported by the other linear or rod-shaped portion.

As illustrated in FIG. 12, the interval between the two sides rotatably supporting the first roller 10a1 and the second roller 10a2 coincides with the interval between the two flow passage walls located on the outside of the three flow passage walls. That is, the interval is the same as the interval between the flow passage wall dividing the first branching flow passage 2a and the third branching flow passage 2c and the flow passage wall dividing the fourth branching flow passage 2d and the second branching flow passage 2b. In addition, the length in the longitudinal direction of the first roller 10a1 and the second roller 10a2 is at least longer than the interval from the left side end portion (central side end portion of the branching member) of the three flow passage walls to the left side surface of the branching member (flexible branching section) 2′. In addition, an area of the flat plate-shaped support member (not illustrated) is larger than an area of at least the branching member (flexible branching section) 2′. The flat plate-shaped support member has an area which covers the area for locating the connection member 10b. In this manner, the branching member (flexible branching section) 2′ can be placed (located) on the flat plate-shaped support member. The connection member 10b which rotatably supports and connects the first roller 10a1 and the second roller 10a2 is movable in one direction while pinching the branching member (flexible branching section) 2′ with the above-described support member. A force or a load is always applied to the first roller 10a1 and the second roller 10a2 in a direction in which the branching member (flexible branching section) 2′ is squeezed.

In a state illustrated in the left drawing in FIG. 12, in cooperation with the second roller 10a2 and the flat plate-shaped support member, the branching member (flexible branching section) 2′ is brought into a state squeezed (closed) from the central portion side end portion of the flow passage wall dividing the third branching flow passage 2c and the fourth branching flow passage 2d to the left side surface of the branching member (flexible branching section) 2′. In addition, in cooperation with the first roller 10a1 and the flat plate-shaped support member, the upper side portion of the branching member (flexible branching section) 2′ is in a squeezed (closed) state. In this manner, the inflow passage 4 and the entry-side end portion 6a of the circulation flow passage 6 communicate (are continuous) with each other via the first branching flow passage 2a and the third branching flow passage 2c, and the exit-side end portion 6b of the circulation flow passage 6 and the outflow passage 5 communicate (are continuous) with each other via the fourth branching flow passage 2d and the second branching flow passage 2b, thereby forming the open flow passage (open system) and allowing the second communication state. At this time, the liquid such as the culture solution flows back on the left side surface of the branching member (flexible branching section) 2′.

In a state illustrated in the left drawing in FIG. 12, the connection member 10b which rotatably supports and connects the first roller 10a1 and the second roller 10a2 is moved in the horizontal direction (direction from the left side toward the lower side in FIG. 12) by an actuator (not illustrated), for example. This state is illustrated in the right drawing in FIG. 12. As illustrated in the right drawing in FIG. 12, in cooperation with the first roller 10a1 and the flat plate-shaped support member, the range from the central portion side end portion of the flow passage wall dividing the second branching flow passage 2b and the fourth branching flow passage 2d to the left side surface of the branching member (flexible branching section) 2′ is squeezed (closed). At the same time, in cooperation with the second roller 10a2 and the flat-plate-shaped support member, the range from the central portion side end portion of the flow passage wall dividing the first branching flow passage 2a and the third branching flow passage 2c to the left side surface of the branching member (flexible branching section) 2′ is squeezed (closed). In this manner, the exit-side end portion 6b of the circulation flow passage 6 and the entry-side end portion 6a of the circulation flow passage 6 communicate (are continuous) with each other via the fourth branching flow passage 2d and the third branching flow passage 2c, thereby forming the circulation flow passage (closed system) and allowing the first communication state. At this time, the liquid such as the culture solution flows back on the left side face of the branching member (flexible branching section) 2′.

According to the present embodiment, in the second communication state and the first communication state, the liquid such as the culture solution collides once with the left side surface of the branching member (flexible branching section) 2′, and thereafter, flows back. Accordingly, although it is not necessarily desirable from a viewpoint of a streamline, all of the flow passage directions of the first branching flow passage 2a to the fourth branching flow passage 2d are aligned with each other. Therefore, the inflow passage 4 is easily connected to each branching flow passage. In addition, as illustrated in FIG. 12, flow passage widths of the four branching flow passages (2a to 2d) are equal to each other. Accordingly, only two rollers functioning as pressing portions can be provided. Compared to Embodiment 3, the number of components can be reduced.

### Embodiment 5

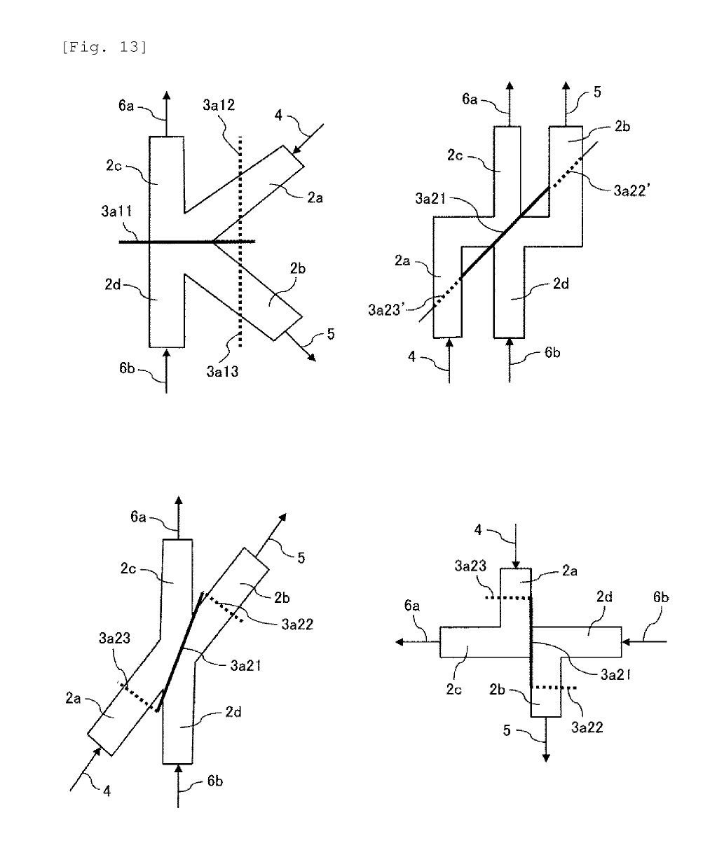

FIG. 13 is a schematic configuration diagram of the flow passage module according to Embodiment 5 serving as another embodiment of the present invention. The present embodiment is different from Embodiment 1 to Embodiment 4 described above in an arrangement relationship or a connection relationship of the first branching flow passage 2a to the fourth branching flow passage 2d which configure the branching member (flexible branching section) 2.

The other configuration elements are the same as those of Embodiment 1. Thus, hereinafter, repeated description in Embodiment 1 will be omitted.

The flow passage module 1 illustrated in the upper left drawing in FIG. 13 includes the branching member (flexible branching section) 2 and the communication state switching part 3 illustrated in FIG. 5 according to Embodiment 1. In the branching member (flexible branching section) 2, the third branch flow passage 2c connected to the entry-side end portion 6a of the circulation flow passage 6 (not illustrated) and the fourth branching flow passage 2d connected to the exit-side end portion 6b of the circulation flow passage 6 face and communicate with each other, and the third branching flow passage 2c and the fourth branching flow passage 2d are located linearly. In contrast, the first branching flow passage 2a connected to the inflow passage 4 is joined to a substantially central portion of the third branching flow passage 2c and the fourth branching flow passage 2d, which are linearly located at a predetermined angle (acute angle). In addition, similarly, the second branching flow passage 2b connected to the outflow passage 5 is joined to a substantially central portion of the third branching flow passage 2c and the fourth branching flow passage 2d, which are linearly located at a predetermined angle (acute angle). Then, the first branching flow passage 2a and the second branching flow passage 2b are located on the same side in a plan view with respect to the third branching flow passage 2c and the fourth branching flow passage 2d which are linearly located. In addition, the opening/closing member configuring the communication state switching part 3 is configured to include the pinch member (pressing member) 3a1 illustrated in FIG. 5 according to Embodiment 1 and the support member 3b′ which always maintains a stationary state. In the second communication state, the branching member (flexible branching section) 2 is pinched (squeezed) by the first pressing portion 3a11 configuring the pinch member (pressing member) 3a1 and the NC-side member 3b1 (not illustrated in FIG. 13) configuring the support member 3b′. The inflow passage 4 and the entry-side end portion 6a of the circulation flow passage 6 communicate with each other via the first branching flow passage 2a and the third branching flow passage, and the exit-side end portion 6b of the circulation flow passage 6 and the outflow passage 5 communicate with each other via the fourth branching flow passage 2d and the second branching flow passage 2b, thereby forming the open flow passage (open system). At this time, the liquid such as the culture solution flows through the first branching flow passage 2a, flows back at an acute angle, and flows through the third branching flow passage 2c. Therefore, it is desirable that an angle formed between the first branching flow passage 2a and the third branching flow passage is appropriately set to fall within a range which does not increase flow passage resistance as much as possible. It is desirable to similarly set an angle between the second branching flow passage 2b and the fourth branching flow passage 2d. On the other hand, in the first communication state, the first branching flow passage 2a is pinched by the second pressing portion 3a12 configuring the pinch member (pressing member) 3a1 and the NO-side member 3b2′ (not illustrated in FIG. 13), and the second branching flow passage 2b is pinched by the third pressing portion 3a13 and the NO-side member 3b2′. In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d which are linearly located, thereby forming the circulation flow passage (closed system). Therefore, the liquid smoothly flows in the circulation flow passage.

The flow passage module 1 illustrated in the upper right drawing in FIG. 13 includes a configuration obtained by partially modifying the branching member (flexible branching section) 2 and the communication state switching part 3 illustrated in FIG. 6 according to Embodiment 1. In the branching member (flexible branching section) 2, the first branching flow passage 2a and the fourth branching flow passage 2d are parallel to each other, and are located apart from each other at a predetermined interval. In addition, the second branching flow passage 2b and the third branching flow passage 2c are parallel to each other, and are located apart from each other at a predetermined interval, and the third branching flow passage 2c and the fourth branching flow passage 2d are linearly located. Since the branching member (flexible branching section) 2 has this shape, the first pressing portion 3a21, the second pressing portion 3a22′, and the third pressing portion 3a23′ which configure the pinch member (pressing member) can be linearly located in a vertical projection plane. The second pressing portion 3a22′ and the third pressing portion 3a23′ are located below the first pressing portion 3a21 at a predetermined interval. In this manner, three locations of the branching member (flexible branching section) 2 can be pinched by one pinch member (pressing member). Therefore, compared to Embodiment 1, it is possible to simplify the shape of the pinch member or to reduce the configuration elements of the pinch member.

The flow passage module 1 illustrated in the lower left drawing in FIG. 13 includes the branching member (flexible branching section) 2 and the communication state switching part 3 illustrated in FIG. 6 according to Embodiment 1. The branching member (flexible branching section) 2 includes a configuration in which the first branching flow passage 2a and the second branching flow passage 2b are linearly located, and the third branching flow passage 2c and the fourth branching flow passage 2d are linearly located so that all of these are joined to each other at a predetermined angle. In other words, in a case where the branching member (flexible branching section) 2 is based on the third branching flow passage 2c and the fourth branching flow passage 2d which are linearly located, the branching member (flexible branching section) 2 is provided with a shape in which the first branching the flow passage 2a and the second branching flow passage 2b are inclined and joined to each other. The liquid such as the culture solution can smoothly flow in any case of the second communication state for forming the open flow passage (open system) by causing the first pressing portion 3a21 and the NC-side member 3b11 (not illustrated in FIG. 13) to pinch the branching member (flexible branching section) 2 and the first communication state for forming the circulation flow passage (the closed system) by causing the second pressing portion 3a22 and the first NO side member 3b21 (not illustrated in FIG. 13) to pinch the second branching flow passage 2b and by causing the third pressing portion 3a23 and the second NO-side member 3b22 to pinch the first branching flow passage 2a.

The flow passage module 1 illustrated in the lower right drawing in FIG. 13 includes the branching member (flexible branching section) 2 and the communication state switching part 3 illustrated in FIG. 6 according to Embodiment 1. The branching member (flexible branching section) 2 is provided with a shape in which the third branching flow passage 2c and the fourth branching flow passage 2d are linearly located, and the first branching flow passage 2a and the second branching flow passage 2b which are located so as to be orthogonal to the third branching flow passage 2c and the fourth branching flow passage 2d are arranged to be offset. In other words, the first branching flow passage 2a and the second branching flow passage 2b are located diagonally opposite to each other. The communication state switching part 3 illustrated in FIG. 6 is applied to the branching member (flexible branching section) 2 having this shape. Accordingly, in any case of the second communication state and the first communication state, the liquid such as the culture solution can be supplied into the branching member (flexible branching section) 2 without any remaining liquid.

### Embodiment 6

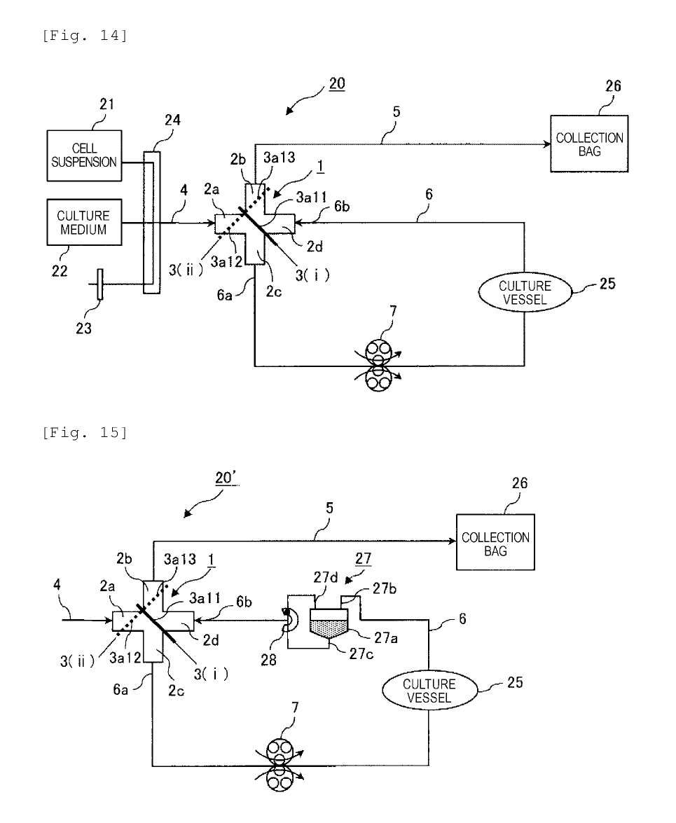

FIG. 14 is an overall schematic configuration diagram of a cell culture apparatus having the flow passage module according to Embodiment 6 serving as another embodiment of the present invention, and FIG. 15 is a view illustrating a modification example of the cell culture apparatus illustrated in FIG. 14. Hereinafter, a configuration of the flow passage module 1 illustrated in FIG. 7 according to Embodiment 1 above will be described as an example. Even when either the configuration of the flow passage module 1 illustrated in FIG. 8 or the configuration of the flow passage module described according to Embodiment 2 to Embodiment 5 is adopted, the same is applied thereto. In FIGS. 14 and 15, the same reference numerals will be given to configuration elements which are the same as those described according to Embodiment 1 above. Thus, hereinafter, repeated description in Embodiment 1 will be omitted.

As illustrated in FIG. 14, a cell culture apparatus 20 includes a supply bag which accommodates a cell suspension 21, a supply bag which accommodates a culture medium 22, a HEPA filter 23, a flow passage switching part 24, the flow passage module 1, the circulation flow passage 6, a pump 7, for example, such as a squeezing pump installed in the circulation flow passage 6, a culture vessel 25 installed in the circulation flow passage 6 on the downstream side of the pump 7, and a collection bag 26 for collecting the culture medium or the culture solution subjected to the culture. The cell culture apparatus 20 performs culture by automatically performing cell seeding and culture medium exchange automatically. The cell suspension 21 or the culture medium 22 is introduced into the circulation flow passage 6 via the flow passage module 1 as will be described in detail later, and is circulated inside the circulation flow passage 6 where the culture vessel 25 is installed, thereby forming a closed system. In this way, the culture is performed inside the circulation flow passage 6 forming the closed system. In this manner, it is possible to prevent contamination from the outside, and to perform highly reliable culture.

The cell suspension 21 or the culture medium 22 is introduced into the inflow passage 4, and is selectively supplied by the flow passage switching part 24. In addition, the HEPA filter 23 can be selectively connected to the inflow passage 4 by the flow passage switching part 24, and air permeating the HEPA filter 23 can push out the liquid remaining in the flow passage or flowing in the flow passage. The inflow passage 4 is connected to the first branching flow passage 2a of the branching member (flexible branching section) 2. The outflow passage 5 whose one end is connected to the collection bag 26 is connected to the second branching flow passage 2b of the branching member (flexible branching section) 2. In addition, the entry-side end portion 6a of the circulation flow passage 6 is connected to the third branching flow passage 2c of the branching member (flexible branching section) 2, and the exit side end 6b of the circulation flow passage 6 is connected to the fourth branching flow passage 2d of the branching member (flexible branching section) 2. When the cell seeding or the culture medium exchange is performed, the coil 3f configuring the communication state switching part 3 illustrated in FIG. 4 is brought into a state where no power is supplied, thereby squeezing (pinching) the branching member (flexible branching section) 2 in the direction of 3(i). That is, the branching member (flexible branching section) 2 is pinched in a planarly diagonal line shape by the first pressing portion 3a11 configuring the pinch member (pressing member) 3a1 illustrated in FIG. 5 and the NC-side member 3b1 (not illustrated in FIG. 14) configuring and the support member 3b′ so as to cross the corner portion where the first branching flow passage 2a and the second branching flow passage 2b are joined to each other and the corner portion where the third branching flow passage 2c and the fourth branching flow passage 2d are joined to each other. In this manner, the inflow passage 4 and the entry-side end portion 6a of the circulation flow passage 6 communicate with each other via the first branching flow passage 2a and the third branching flow passage 2c, and the exit-side end portion 6b of the circulation flow passage 6 and the outflow passage 5 communicate with each other via the fourth branching flow passage 2d and the second branching flow passage 2b, thereby forming the open flow passage (open system) and allowing the second communication state.

Next, the pump 7 installed in the circulation flow passage 6 is driven, thereby causing the branching member (flexible branching section) 2 to internally have negative pressure. Thus, the cell suspension 21 or the culture medium 22 is aspirated into the first branching flow passage 2a from the inflow passage 4. The cell suspension 21 or the culture medium 22 aspirated into the first branching flow passage 2a is introduced into the entry-side end portion 6a of the circulation flow passage 6 via the third branching flow passage 2c, flows into the circulation flow passage 6, and flows into the fourth branching flow passage 2d from the exit-side end portion 6b of the circulation flow passage 6 via the culture vessel 25 installed in the circulation flow passage 6. Thereafter, the cell suspension 21 or the culture medium 22 flows through the fourth branching flow passage 2d and the second branching flow passage 2b, flows out to the outflow passage 5, and is supplied to the collection bag 26. In the case of stationary culture, the second communication state is continuously maintained by the above-described communication state switching part 3.

On the other hand, in a case where the cell suspension 21 or the culture medium 22 is circulated inside the circulation flow passage 6 for a certain purpose, the power is supplied to the coil 3f illustrated in FIG. 4 described above, thereby pinching the branching member (flexible branching section) 2 in the direction of 3(ii). That is, the first branching flow passage 2a is pinched by the second pressing portion 3a12 configuring the pinch member (pressing member) 3a1 illustrated in FIG. 5 and the NO-side member 3b2′ (not illustrated in FIG. 14), and the second branching flow passage 2b is pinched by the third pressing portion 3a13 and the NO-side member 3b2′. In this manner, the entry-side end portion 6a of the circulation flow passage 6 and the exit-side end portion 6b of the circulation flow passage 6 communicate (are continuous) with each other via the third branching flow passage 2c and the fourth branching flow passage 2d, thereby forming the circulation flow passage (closed system) and switching the second communication state to the first communication state. For example, in the first communication state, a fluid shearing force can be applied by circulating the cell suspension 21 or the culture medium 22 inside the circulation flow passage 6.

FIG. 15 is an overall schematic configuration diagram of a cell culture apparatus 20′ having a buffer tank 27 on the downstream side of the culture vessel 25 in the circulation flow passage 6. In the circulation flow passage 6, a flow passage volume is uniquely determined by the flow passage diameter (inner diameter) and the flow passage length. Thus, it is difficult to flexibly handle a culture amount. Therefore, a configuration is adopted in which the buffer tank 27 is installed in the circulation flow passage 6 so that the culture amount can be flexibly changed. The buffer tank 27 is installed on the downstream side of the culture vessel 25 in the circulation flow passage 6, and is configured to include a tank housing 27a, an inflow port 27b, an outflow port 27c, and an air discharge port 27d. The outflow port 27c is disposed in the lower portion of the tank housing 27a, and the air discharge port 27d is disposed in the upper portion of the tank housing 27a. In a case where the cell suspension 21 or the culture medium 22 is supplied to the circulation flow passage 6 via the flow passage module 1, the cell suspension 21 or the culture medium 22 is caused to flow from the outflow port 27c to the downstream side of the circulation flow passage 6. On the other hand, in a case where the air fully filled in the tank housing 27a is discharged, the air is pushed to the downstream side of the circulation flow passage 6 via the air discharge port 27d, and the communication state switching part 3 switches the first communication state to the second communication state, thereby discharging the air via the fourth branching flow passage 2d, the second branching flow passage 2b of the branching member (flexible branching section) 2 and the outflow passage 5. The outflow port 27c and the air discharge port 27d are selectively switched therebetween by a switching valve 28. An installation position of the inflow port 27b is not necessarily limited to the upper portion of the tank housing 27a.

In the present embodiment, the cell culture apparatus 20 is configured to have the HEPA filter 23. However, the HEPA filter 23 is not indispensable. For example, instead of the HEPA filter 23, a configuration may be adopted which has a bag for accommodating system water such as pure water. In the second communication state, the system water is caused to flow into the circulation flow passage 6 via the branching member (flexible branching section) 2, and is discharged via the outflow passage 5, thereby enabling the culture medium exchange to be suitably performed.

According to the present embodiment, it is possible to realize the cell culture apparatus which can supply the cell suspension or the culture medium into the circulation flow passage while preventing bubbles from being mixed into the circulation flow passage.

In addition, since the buffer tank is installed in the circulation flow passage, it is possible to obtain a desired culture amount.

### Embodiment 7

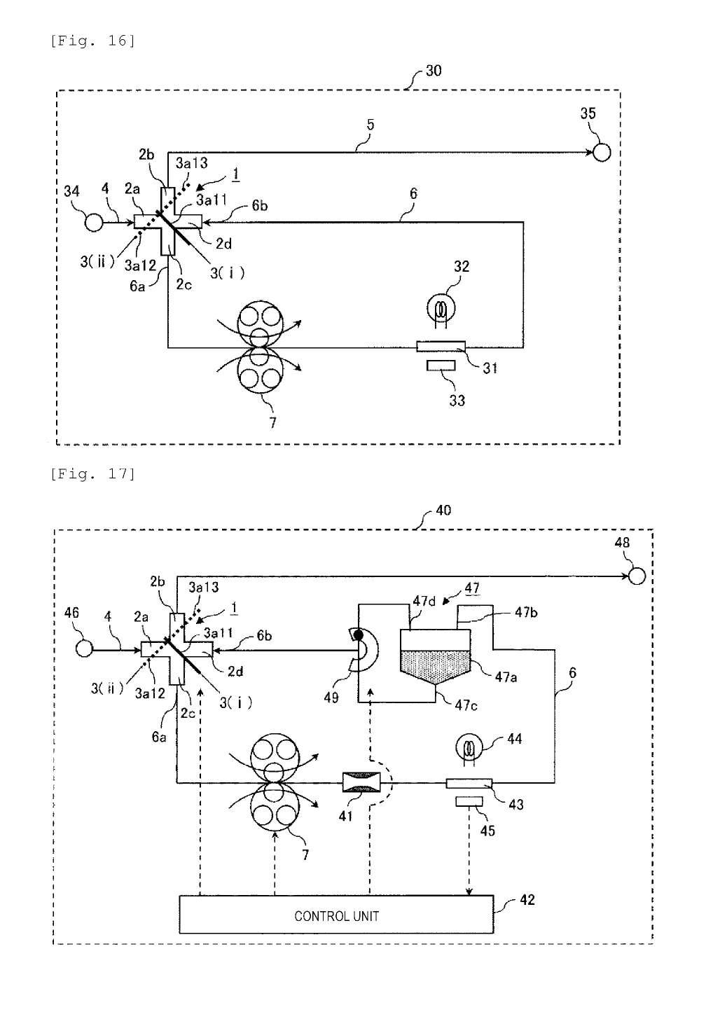

FIG. 16 is an overall schematic configuration diagram of a turbidity meter having the flow passage module according to Embodiment 7 serving as another embodiment of the present invention. Hereinafter, a configuration of the flow passage module 1 illustrated in FIG. 7 according to Embodiment 1 above will be described as an example. Even when either the configuration of the flow passage module 1 illustrated in FIG. 8 or the configuration of the flow passage module described according to Embodiment 2 to Embodiment 5 is adopted, the same is applied thereto. In FIG. 16, the same reference numerals will be given to configuration elements which are the same as those described according to Embodiment 1 above. Thus, hereinafter, repeated description in Embodiment 1 will be omitted.

As illustrated in FIG. 16, a turbidity meter 30 includes an inflow port 34 which introduces a liquid having unknown turbidity, the flow passage module 1, the circulation flow passage 6, the pump 7, for example, such as a squeezing pump installed in the circulation flow passage 6, a flow cell 31 installed on the downstream side of the pump 7 in the circulation flow passage 6, alight source 32 for emitting light to the liquid flowing inside the flow cell 31, a detector 33 installed on a side opposite to the light source 32 across the flow cell 31, and the outflow passage 5 which supplies the liquid whose turbidity is measured to the outflow port 35.

First, the branching member (flexible branching section) 2 is squeezed (pinched) in the direction of 3(i) by bringing the coil 3f configuring the communication state switching part 3 illustrated in FIG. 4 into a state where no power is supplied thereto. That is, the branching member (flexible branching section) 2 is pinched in a planarly diagonal line shape by the first pressing portion 3a11 configuring the pinch member (pressing member) 3a1 illustrated in FIG. 5 and the NC-side member 3b1 (not illustrated in FIG. 16) configuring the support member 3b′ so as to cross the corner portion where the first branching flow passage 2a and the second branching flow passage 2b are joined to each other and the corner portion where the third branching flow passage 2c and the fourth branching flow passage 2d are joined to each other. In this manner, the inflow passage 4 and the entry-side end portion 6a of the circulation flow passage 6 communicate with each other via the first branching flow passage 2a and the third branching flow passage 2c, and the exit-side end portion 6b of the circulation flow passage 6 and the outflow passage 5 communicate with each other via the fourth branching flow passage 2d and the second branching flow passage 2b, thereby forming the open flow passage (open system) is formed and allowing the second communication state.

Next, the pump 7 installed in the circulation flow passage 6 is driven, thereby causing the branching member (flexible branching section) 2 to internally have negative pressure. The liquid having the unknown turbidity which is introduced from the inflow port 34 is aspirated into the first branching flow passage 2a from the inflow passage 4. The liquid having the unknown turbidity which is aspirated into the first branching flow passage 2a is introduced to the entry-side end portion 6a of the circulation flow passage 6 via the third branching flow passage 2c, flows through the circulation flow passage 6, and flows into the flow cell 31 installed on the downstream side of the pump 7. At this time, the light is emitted from the light source 32 toward the liquid flowing into the flow cell 31. The transmitted light and/or the scattered light is received by the detector 33, and the turbidity is measured, based on light intensity of the transmitted light and/or the scattered light. Thereafter, the liquid whose turbidity is measured flows from the exit-side end portion 6b of the circulation flow passage 6, and is discharged from the outflow port 35 via the fourth branching flow passage 2d and the second branching flow passage 2b of the branching member (flexible branching section) 2 and the outflow passage 5.

In some cases, the liquid has low turbidity, or in some cases, the turbidity cannot be measured through single measurement due to disturbance noise such as ambient light. In this case, the power is supplied to the coil 3f illustrated in FIG. 4 described above, thereby pinching the branching member (flexible branching section) 2 in the direction of 3 (ii). That is, the first branching flow passage 2a is pinched by the second pressing portion 3a12 configuring the pinch member (the pressing member) 3a1 illustrated in FIG. 5 and the NO-side member 3b2 (not illustrated in FIG. 16), and the second branching flow passage 2b is pinched by the third pressing portion 3a13 and the NO-side member 3b2′. In this manner, the entry-side end portion 6a of the circulation flow passage 6 and the exit-side end portion 6b of the circulation flow passage 6 communicate with each other via the third branching flow passage 2c and the fourth branching flow passage 2d, thereby forming the circulation flow passage (closed system) and switching the second communication state to the first communication state. The liquid flows into the circulation flow passage 6, and the liquid flows into the flow cell 31 again. The light is emitted again from the light source 32 toward the liquid flowing into the flow cell 31, and the detector 33 receives the transmitted light and/or the scattered light. In this manner, the turbidity is measured, based on the light intensity of the transmitted light and/or the scattered light. In a case where the turbidity is measured, the communication state switching part 3 switches the first communication state to the second communication state, and the liquid whose turbidity is measured is discharged from the outflow port 35 via the fourth branching flow passage 2d, the second branching flow passage 2b of the branching member (flexible branching section) 2 and the outflow passage 5.

According to the present embodiment, the liquid having the unknown turbidity is introduced into the circulation flow passage by causing the communication state switching part to switch the first communication state to the second communication state. In this manner, it is possible to easily measure the turbidity by using a turbidity detection mechanism configured to include the light source and the detector.

In addition, even in a case where the liquid has the low turbidity or even in a case where the turbidity cannot be measured through single measurement due to disturbance noise such as ambient light, the second communication state is switched to the first communication state by the communication state switching part so that the liquid is circulated inside the circulation flow passage. In this manner, it is possible to reliably measure the turbidity.

### Embodiment 8

FIG. 17 is an overall schematic configuration diagram of a cell dispersing device having the flow passage module according to Embodiment 8 serving as another embodiment of the present invention. Hereinafter, a configuration of the flow passage module 1 illustrated in FIG. 7 according to Embodiment 1 above will be described as an example. Even when either the configuration of the flow passage module 1 illustrated in FIG. 8 or the configuration of the flow passage module described according to Embodiment 2 to Embodiment 5 is adopted, the same is applied thereto. In FIG. 17, the same reference numerals will be given to configuration elements which are the same as those described according to Embodiment 1 above. Thus, hereinafter, repeated description in Embodiment 1 will be omitted.

As illustrated in FIG. 17, a cell dispersing device 40 includes an inflow port 46 which introduces the cell suspension having unknown cell dispersion degree, the flow passage module 1, the circulation flow passage 6, the pump 7, for example, such as a squeezing pump installed in the circulation flow passage 6, an orifice 41 located on the downstream side of the pump 7 in the circulation flow passage 6, a flow cell 43 installed on the downstream side of the orifice 41 in the circulation flow passage 6, a light source 44 for emitting the light to the cell suspension flowing into the flow cell 43, a detector 45 installed on a side opposite to the light source 44 across the flow cell 43, a buffer tank 47 installed on the downstream side of the flow cell 43 in the circulation flow passage 6, an outflow port 48 for discharging the cell suspension whose cells are uniformly dispersed, and a control unit 42. The cell dispersing device 40 has a function to fetch the cell suspension having unknown cell dispersion degree from the inflow port 46, to internally disperse the cell aggregate, and to discharge the cell suspension whose cell is uniformly dispersed, from the outflow port 48. The orifice 41 installed on the downstream side of the pump 7 in the circulation flow passage 6 forms a flow passage narrowing portion. The orifice 41 applies a strong shearing force to the internally flowing cell suspension by rapidly changing a cross-sectional area of the flow passage, thereby promoting dispersion of the cell aggregate. In view of the fact that a size of the cell is generally and approximately 10 μm, it is preferable that the diameter (cross-sectional diameter) of the orifice 41 falls within a range of 0.5 mm to 1 mm, since the cell aggregate can be efficiently dispersed. In addition, based on the size or adhesiveness of the cell, the orifice diameter suitable for each cell may be changed. If an inexpensive resin material is used for the orifice 41, if necessary, each flow passage is disposable, that is, the orifice is disposable. Accordingly, from a viewpoint of preventing contamination, it is desirable to use the disposable orifice.

In the flow cell 43 disposed on the downstream side of the orifice 41 in the circulation flow passage 6, when the cell suspension flows into the flow cell, the light intensity is measured as data relating to the dispersion degree of the cell aggregate. The light is emitted from the light source 44 toward the flow cell 43, and the transmitted light and/or the scattered light is detected by the detector 45. In other words, the light source 44, the flow cell 43, and the detector 45 configure a cell dispersion degree measuring instrument. The light quantity of the transmitted light and/or the scattered light observed from the flow cell 43 varies depending on a change in the cell dispersion degree of the cell suspension. Therefore, a temporal change of the light intensity detected by the detector 45 is focused on. In this manner, it is possible to determine whether the cell is sufficiently dispersed, based on the fact that the change amount of the light intensity value decreases so as to converge to a fixed value (preferably, a predetermined target value). Based on the light intensity data obtained by the detector 45, the control unit 42 determines whether or not the cell dispersion degree reaches a predetermined dispersion degree. In a case where the cell dispersion degree reaches the predetermined dispersion degree, the control unit 42 continues to drive the pump 7. The shearing force applied to the cell suspension may be changed by changing liquid supply speed of the pump 7.

In this way, in order to obtain the cell suspension in which the cell aggregate contained in the cell suspension is dispersed and the cells are uniformly distributed inside the circulation flow passage 6, the coil 3f configuring the communication state switching part 3 illustrated in FIG. 4 is first brought into a state where no power is supplied thereto, thereby squeezing (pinching) the branching member (flexible branching section) 2 in the direction of 3(i). That is, the branching member (flexible branching section) 2 is pinched in a planarly diagonal line shape by the first pressing portion 3a11 configuring the pinch member (pressing member) 3a1 illustrated in FIG. 5 and the NC-side member 3b1 (not illustrated in FIG. 17) configuring the support member 3b′ so as to cross the corner portion where the first branching flow passage 2a and the second branching flow passage 2b are joined to each other and the corner portion where the third branching flow passage 2c and the fourth branching flow passage 2d are joined to each other. In this manner, the inflow passage 4 and the entry-side end portion 6a of the circulation flow passage 6 communicate with each other via the first branching flow passage 2a and the third branching flow passage 2c, and the exit-side end portion 6b of the circulation flow passage 6 and the outflow passage 5 communicate with each other via the fourth branching flow passage 2d and the second branching flow passage 2b, thereby forming the open flow passage (open system) and allowing the second communication state.

Next, the pump 7 installed in the circulation flow passage 6 is driven, thereby causing the branching member (flexible branching section) 2 to internally have negative pressure. The cell suspension introduced from the inflow port 46 is aspirated into the first branching flow passage 2a from the inflow passage 4. The cell suspension aspirated into the first branching flow passage 2a is introduced into the entry-side end portion 6a of the circulation flow passage 6 via the third branching flow passage 2c, flows into the circulation flow passage 6, and flows into the orifice 41 and the flow cell 31 which are installed on the downstream side of the pump 7. A configuration and an operation of the buffer tank 47 is the same as that according to Embodiment 6 illustrated in FIG. 15. Thus, description thereof will be omitted.

As a method of measuring the cell dispersion degree, as described above, it is particularly preferable to employ the following method. If the light is emitted from the light source 44 toward the flow cell 43 and the transmitted light and/or the scattered light is detected by the detector 45, it is possible to measure the cell dispersion degree while maintaining a flowing state of the cell suspension. However, the method of measuring the cell dispersion degree is not limited thereto, and other methods may be employed. For example, any observation window may be disposed in the circulation flow passage 6, and an image (a still image or a moving image) may be captured using a microscope equipped with a CCD camera, thereby calculating the cell dispersion degree from the acquired image. In order to measure the cell dispersion degree in the flowing state of the cell suspension, the process needs to be performed on a real-time basis. However, if this high-speed image processing is available, the above-described method can be employed as a cell dispersion degree measurement method instead of light intensity measurement.

As a material of the tube configuring the circulation flow passage 6, it is preferable to use a material which has no influence on the cells or has extremely small influence. As an example of this material, a medical silicone tube may be used. In addition, the flow cell 43 may be made of glass. However, if an inexpensive resin is used, it is more preferable, since the tube including the circulation flow passage 6 which passes through the cells once is easily disposable.

According to the present embodiment, the communication state switching part configuring the flow passage module switches the first communication state to the second communication state. In this manner, the cell aggregate contained in the cell suspension can be easily dispersed, and the cell suspension whose cells are uniformly dispersed can be obtained.

### Embodiment 9

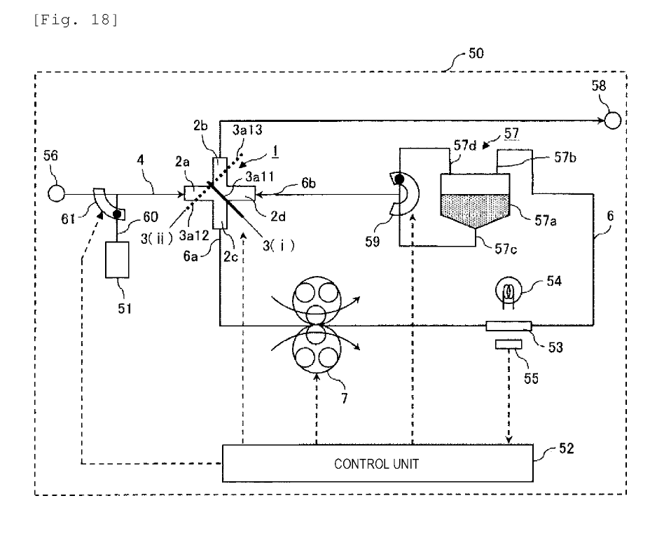

FIG. 18 is an overall schematic configuration diagram of a cell number adjustment device having the flow passage module according to Embodiment 9 serving as another embodiment of the present invention. Hereinafter, a configuration of the flow passage module 1 illustrated in FIG. 7 according to Embodiment 1 above will be described as an example. Even when either the configuration of the flow passage module 1 illustrated in FIG. 8 or the configuration of the flow passage module described according to Embodiment 2 to Embodiment 5 is adopted, the same is applied thereto. In FIG. 18, the same reference numerals will be given to configuration elements which are the same as those described according to Embodiment above. Thus, hereinafter, repeated description in Embodiment 1 will be omitted.

As illustrated in FIG. 18, a cell number adjustment device 50 includes an inflow port 56 which introduces the cell suspension having unknown cell number concentration (the number of cells contained per unit amount of the cell suspension) containing cells at high concentration, a dilution vessel 51 for adjusting the cell number concentration by adding a diluent to the introduced cell suspension, the flow passage module 1, the circulation flow passage 6, the pump 7, for example, such as a squeezing pump installed in the circulation flow passage 6, a flow cell 53 installed on the downstream side of the pump 7 in the circulation flow passage 6, a light source 54 for emitting the light to the cell suspension flowing into the flow cell 53, a detector 55 installed on a side opposite to the light source 54 across the flow cell 53, a buffer tank 57 installed on the downstream side of the flow cell 53 in the circulation flow passage 6, an outflow port 58 for discharging the cell suspension whose cell number is adjusted, and a control unit 52.

The cell number adjustment device 50 has a function to fetch the cell suspension having unknown cell number concentration (the number of cells contained per unit amount of the cell suspension) containing cells at high concentration from the inflow port 56, to internally adjust the concentration, and to discharge the cell suspension containing cells at desired cell number concentration lower than the cell number concentration in the cell suspension flowing from the inflow port 56, from the outflow port 58. A flow passage system including the circulation flow passage 6 is established between the inflow port 56 and the outflow port 58. The pump 7 serving as a liquid supply pump for causing the cell suspension to flow into the flow passage is provided, and the control unit 52 controls at least the pump 7.

When the cell suspension flows into the flow cell 53 disposed on the downstream side of the pump 7 in the circulation flow passage 6, the light intensity is measured as data relating to the cell number concentration per unit amount. The light is emitted from the light source 54 toward the flow cell 53, and the transmitted light and/or the scattered light is detected by the detector 55. In other words, the light source 54, the flow cell 53, and the detector 55 configure a cell number measuring instrument. A relationship between the intensity of the transmitted light or the scattered light detected by the detector 55 and the number of cells is separately obtained in advance, and the cell number concentration is calculated, based on the relationship between the intensity of the transmitted light or the scattered light and the number of cells and the light intensity detected by the detector 55. For example, the relationship between the intensity of the transmitted light or the scattered light and the number of cells can be determined by preparing several cell suspensions having known concentration of culture scheduled cells, measuring light intensity for each cell suspension, and preparing a calibration curve from the obtained result. A flow rate of the cell suspension passing through the flow cell 53 can be obtained, based on the amount fetched from the inflow port 56 or based on the volume or the cross sectional area of the flow cell 53 and the liquid supply speed of the pump 7. The amount of the required diluent is determined, based on the cell number concentration and the amount of the cell suspension.

According to the cell number concentration measurement based on the light intensity, the cell number concentration can be calculated in a flowing state of the cell suspension. In a case where the cell number concentration is calculated in the flowing state of the cell suspension, the detector 55 may continuously measure the light intensity without any interruption, or may intermittently measure the light intensity, that is, at an interval between the measurements. It is preferable to measure the light intensity at each constant interval. In the cell number adjustment device 50 according to the present embodiment, another calculation method may be used so as to calculate the cell number concentration.

The inflow passage 4 is partially branched, and is connected to the branching flow passage 60. A switching valve 61 is disposed in the branching portion. The switching valve 61 can switch between the branching flow passage 60 and the inflow passage 4. As the switching valve 61, it is preferable to use a pinch valve. The pinch valve controls the flow by squeezing (pinching) the flow passage formed of an elastic material from the outside, and does not directly touch the fluid. Accordingly, the cell suspension and the pinch valve itself are not contaminated, and the cell suspension can be controlled. The switching valve 61 has a function to switch between two flow passages and can be realized by combining two pinch valves with each other. However, a universal type may be used which can control the two flow passages so as to be alternately open and closed at the same time by using one actuator. The control unit 52 controls switching of the valves by controlling the actuator disposed in the switching valve 61. The same is applied to other switching valves (to be described later).

The diluent vessel 51 for accommodating the diluent is connected to a front end of the branching flow passage 60. The control unit 52 also controls at least the pump 7, preferably together with the switching valve 61. The diluent is added to the fetched cell suspension in accordance with the detection result of the detector 55, and further, and cell suspension and the added diluent are sufficiently stirred so that the cell number concentration becomes uniform. The control of the pump 7 and the switching valve 61 which is performed by the control unit 52 will be described in detail below.

The control unit 52 drives the pump 7 in a state where the switching valve 61 closes the branching flow passage 60 and selects the inflow passage 4, and fetches a stock solution of the cell suspension from the inflow port 56. The fetched cell suspension is transferred to the flow cell 53 directly through the flow passage module 1. At this time, the communication state switching part 3 configuring the flow passage module 1 maintains the second communication state. When the cell suspension flows through the flow cell 53, the light intensity is measured by the detector 55. The control unit 52 calculates the cell number concentration from the measurement result, compares the cell number concentration with a predetermined target value, and thereafter, determines the required amount of the diluent in view of the amount of the fetched stock solution.

The control unit 52 subsequently switches the switching valve 61 to a state where the branching flow passage 60 side is selected, drives the pump 7 for a fixed time, and fetches the diluent from the diluent vessel 51 via the flow passage module 1 into the circulation flow passage 6. The inside of the circulation flow passage 6 is brought into a state where two liquids of the cell suspension having the high cell number concentration before adjustment and the diluent are present in a non-uniform manner. Next, the control unit 52 mixes the two liquids by driving the pump 7. The circulation flow passage 6 includes a sufficient space to hold the cell suspension and diluent, including a space for movement of the two liquids.

The measurement value of the light intensity measurement has a large fluctuation width, since the cell number concentration in the circulation flow passage 6 is not uniform in an initial stage. However, as the pump 7 is driven, the cell number concentration gradually becomes uniform, and the fluctuation width decreases. The measurement value eventually converges to a target value, that is, a value of the light intensity corresponding to the predetermined cell number concentration. Therefore, when a temporal change in the measurement value of the light intensity measurement falls within a range of a predetermined value (target value ±α), preferably when there is no change, the control unit 52 determines that the liquid contained inside the branching member (flexible branching section) 2 is uniform. In a case where the converged value is different from the target value, the control unit 52 may repeat the above-described dilution step again. The cell suspension having desired cell number concentration through the dilution step is discharged from the outflow port 58 by driving the pump 7.

If the cross sectional area of the circulation flow passage 6 is small compared to the amount of the cell suspension to be handled, the movement inside the flow passage takes time, when the liquid repeatedly moves for mixing, thereby imposing burden on the cells. Therefore, it is preferable that the circulation flow passage 6 through which at least the cell suspension flows, more preferably, the cross sectional area of the flow cell 53 has a sufficient size in view of the size of the cell to be handled and the amount of the fetched cell suspension. For example, if the amount of the fetched cell suspension falls within a range of 1 mL to 1,000 mL, as the tube configuring the circulation flow passage 6, it is preferable to use a tube whose diameter is approximately 1 to 10 mm. As the flow cell 53, it is preferable to use a tube of 1 to 10 mm.

As a material of the tube configuring the circulation flow passage 6, it is preferable to use a material which has no influence on the cells or has extremely small influence. As an example of this material, a medical silicone tube may be used. In addition, the flow cell 53 may be made of glass. However, if an inexpensive resin is used, it is more preferable, since the tube including the circulation flow passage 6 which passes through the cells once is easily disposable.

As a method of measuring the cell number concentration, as described above, it is particularly preferable to employ the following method. If the light is emitted from the light source 54 toward the flow cell 53 and the transmitted light and/or the scattered light is detected by the detector 55, it is possible to measure the cell number concentration while maintaining a flowing state of the cell suspension. However, the method of measuring the cell number concentration is not limited thereto, and other methods may be employed. For example, any observation window may be disposed in the circulation flow passage 6, and an image (a still image or a moving image) may be captured using a microscope equipped with a CCD camera, thereby calculating the cell number concentration from the acquired image. In order to measure the cell number concentration in the flowing state of the cell suspension, the process needs to be performed on a real-time basis. However, if this high-speed image processing is available, the above-described method can be employed as a cell number concentration measurement method instead of light intensity measurement.

The cell suspension is precipitated if the cell suspension is stationary. Accordingly, the cell number adjustment device 50 according to the present embodiment may be used as a device which not only dilutes the cell suspension by adding the diluent but also simply stirs the cell suspension.

If the cell dispersing device 40 and the cell number adjustment device 50 are connected to each other, detached cells are dispersed, and the number of cells is adjusted, thereby allowing re-seeding, that is, it is possible to perform subculture. In addition, for example, the branching flow passage and a diluent bag connected to the branching flow passage are further disposed in the flow passage of the cell dispersing device 40, and based on the light intensity data detected by the detector 45 configuring the cell dispersion degree measuring instrument, the cell suspension concentration is determined. A configuration for fetching the required amount of the diluent from the diluent bag is additionally adopted. In this manner, the cell number adjustment device 50 can be omitted.

The present invention is not limited to the above-described embodiments, and includes various modification examples. For example, the above embodiments have been described in detail in order to facilitate the understanding of the present invention, and are not necessarily limited to those including all of the above-described configurations. In addition, the configuration according to one embodiment can be partially substituted with the configuration of another embodiment, and the configuration according to another embodiment can be added to the configuration of one embodiment. In addition, additions, deletions, or substitutions of the configuration of another embodiment can be made for a part of the configuration of each embodiment.

### REFERENCE SIGNS LIST

1: FLOW PASSAGE MODULE,
2, 2′: BRANCHING MEMBER (FLEXIBLE BRANCHING SECTION),
2a: FIRST BRANCHING FLOW PASSAGE,
2b: SECOND BRANCHING FLOW PASSAGE,
2c: THIRD BRANCHING FLOW PASSAGE,
2d: FOURTH BRANCHING FLOW PASSAGE,
2e: JOINT PORTION,
3, 8, 9, 10: COMMUNICATION STATE SWITCHING PART,
3a, 3a1, 3a2, 8a: PINCH MEMBER (PRESSING MEMBER),
3b, 3b′, 3b″, 8b: SUPPORT MEMBER,
3b1: NORMALLY CLOSED SIDE MEMBER (NC-SIDE MEMBER),
3b2: NORMALLY OPEN SIDE MEMBER (NO-SIDE MEMBER),
3c: MOVABLE IRON CORE,
3d: HOUSING,
3e: SPRING,
3f: COIL,
3g: FIXED IRON CORE,
4: INFLOW PASSAGE,
5: OUTFLOW PASSAGE,
6: CIRCULATION FLOW PASSAGE,
6a: ENTRY-SIDE END PORTION,
6b: EXIT-SIDE END PORTION,
7: PUMP,
8a1, 8a2: PRESSING PORTION,
8c: HINGE,
9a1, 9a2, 9a3, 10a1, 10a2: ROLLER,
9b, 10b: CONNECTION MEMBER,
11a: FIRST FLEXIBLE SHEET,
11b: SECOND FLEXIBLE SHEET,
20, 20′: CELL CULTURE APPARATUS,
21: CELL SUSPENSION,
22: CULTURE MEDIUM,
23: HEPA FILTER,
24: FLOW PASSAGE SWITCHING PART,
25: CULTURE VESSEL,
26: COLLECTION BAG,
27, 47, 57: BUFFER TANK,
27a, 47a, 57a: TANK HOUSING,
27b, 47b, 57b: INFLOW PORT,
27c, 47c, 57c: OUTFLOW PORT,
27d, 47d, 57d: AIR DISCHARGE PORT,
28, 49, 59, 61: SWITCHING VALVE,
30: TURBIDITY METER,
31, 43, 53: FLOW CELL,
32, 44, 54: LIGHT SOURCE,
33, 45, 55: DETECTOR,
34, 46, 56: INFLOW PORT,
35, 48, 58: OUTFLOW PORT,
40: CELL DISPERSING DEVICE,
41: ORIFICE,
42, 52: CONTROL UNIT,
50: CELL NUMBER ADJUSTMENT DEVICE,
51: DILUENT VESSEL,
60: BRANCHING FLOW PASSAGE

1. A flow passage module comprising:
a flexible branching section that includes a first branching flow passage connected to an end portion of an inflow passage of a fluid, a second branching flow passage connected to an end portion of an outflow passage, a third branching flow passage connected to an entry-side end portion of a circulation flow passage, and a fourth branching flow passage connected to an exit-side end portion of the circulation flow passage, and that enables the respective branching flow passages to communicate with each other; and
a communication state switching part that has an opening/closing member which closes or opens a desired branching flow passage of the plurality of branching flow passages, and that moves the opening/closing member in one direction and presses and closes the desired branching flow passage so as to switch a first communication state where the third branching flow passage and the fourth branching flow passage communicate with each other and a second communication state where the first branching flow passage and the third branching flow passage communicate with each other and the second branching flow passage and the fourth branching flow passage communicate with each other.

2. The flow passage module according to claim 1,
wherein the opening/closing member moves in a direction orthogonal to the flexible branching section so as to switch between the first communication state and the second communication state.

3. The flow passage module according to claim 1,
wherein the opening/closing member moves in a direction parallel to the flexible branching section so as to switch between the first communication state and the second communication state.

4. The flow passage module according to claim 1,
wherein the opening/closing member includes a support member which maintains a stationary state and a pressing member which is located on a side opposite to the support member across the flexible branching section, and
wherein the opening/closing member moves the pressing member in one direction, and presses and closes the desired branching flow passage so as to switch between the first communication state and the second communication state.

5. The flow passage module according to claim 4,
wherein the support member is located on a lower surface of the flexible branching section, and has a cross shape or a T-shape in a plan view,
wherein the pressing member has a T-shape in a vertical projection plane, and includes a first pressing portion which extends in one direction while facing the support member, and a second pressing portion which extends in a direction perpendicular to the first pressing portion, and
wherein the second pressing portion has a cross-sectional shape which is inclined upward from a connection portion connected to the first pressing portion, which is bent at a predetermined position, and which is inclined downward at a predetermined angle, and is connected to a hinge disposed in the support member in the connection portion so as to pivot in an arc shape around the hinge serving as a fulcrum.

6. The flow passage module according to claim 3,
wherein the opening/closing member includes a flat plate-shaped support member on which the flexible branching section is placed, and a planarly rectangular connection member which rotatably supports a plurality of rollers positioned on the flexible branching section so as to apply a pressing load to the flexible branching section, and is rotatably supported by two sides where the connection member moves in a direction parallel to the flexible branching member, and where the plurality of rollers are arranged facing each other so as to be orthogonal to a movement direction of the connection member.

7. The flow passage module according to claim 6,
wherein the flexible branching section has a quadrangular shape in a plan view, and the branching flow passage is divided into the first branching flow passage, the third branching flow passage, the fourth branching flow passage, and the second branching flow passage in this order by three flow passage walls extending toward a central portion side of the flexible branching section at equal intervals from one side surface, and
wherein the rollers are respectively and rotatably supported by two sides which are parallel to the flow passage walls in the connection member and which face each other.

8. The flow passage module according to claim 2,
wherein in the flexible branching section, the third branching flow passage and the fourth branching flow passage are linearly located so as to face and communicate with each other, and the first branching flow passage and the second branching flow passage communicate with each other at an angle orthogonal to or at a predetermined angle to the third branching flow passage and the fourth branching flow passage which are linearly located.

9. The flow passage module according to claim 8,
wherein the opening/closing member includes a support member which maintains a stationary state, and a pressing member which moves in a direction orthogonal to the flexible branching section,
wherein the support member has an erected portion which extends in a vertical direction, a first support portion which is bent in an upper end portion of the erected portion and which extends in a horizontal direction, and a second support portion which is positioned below the first support portion at a predetermined interval and which extends from the erected portion in the horizontal direction, and the first support portion and the second support portion are located so as to be orthogonal to each other in a vertical projection plane, and
wherein the pressing member has an erected portion which extends in the vertical direction, a first pressing portion which is positioned in an upper end portion of the erected portion and which extends in the horizontal direction so as to face the first support portion, a second pressing portion which is located on one end side of the first pressing portion and which extends in the horizontal direction so as to face the second support portion, and a third pressing portion which is located on one end side of the first pressing portion and which extends in the horizontal direction so as to face the second support portion.

10. A cell culture apparatus comprising:
an inflow passage through which a cell suspension or a culture medium flows;
a circulation flow passage through which the cell suspension or the culture medium is circulated;
a pump that is installed in the circulation flow passage;
a culture vessel that is installed on a downstream side of the pump and in the circulation flow passage;
a collection bag that is connected to an outflow passage; and
a flow passage module that is connected to the inflow passage, the circulation flow passage, and the outflow passage,
wherein the flow passage module has a flexible branching section that includes a first branching flow passage connected to an end portion of the inflow passage, a second branching flow passage connected to an end portion of an outflow passage, a third branching flow passage connected to an entry-side end portion of the circulation flow passage, and a fourth branching flow passage connected to an exit-side end portion of the circulation flow passage, and that enables the respective branching flow passages to communicate with each other, and a communication state switching part that has an opening/closing member which closes or opens a desired branching flow passage of the plurality of branching flow passages, and that moves the opening/closing member in one direction and presses and closes the desired branching flow passage so as to switch a first communication state where the third branching flow passage and the fourth branching flow passage communicate with each other and a second communication state where the first branching flow passage and the third branching flow passage communicate with each other and the second branching flow passage and the fourth branching flow passage communicate with each other.

11. The cell culture apparatus according to claim 10, further comprising:
a buffer tank that is installed on a downstream side of the culture vessel and in the circulation flow passage,
wherein the buffer tank has an inflow port which introduces the cell suspension or the culture medium flowing in the circulation flow passage, an outflow port which supplies the cell suspension or the culture medium accommodated inside the buffer tank to the circulation flow passage, an air discharge port which discharges air inside the buffer tank to the circulation flow passage, and a switching valve which selectively switches any one of the outflow port and the air discharge port.

12. The cell culture apparatus according to claim 11,
wherein the opening/closing member moves in a direction orthogonal to the flexible branching section, and switches between the first communication state and the second communication state.

13. The cell culture apparatus according to claim 11,
wherein the opening/closing member moves in a direction parallel to the flexible branching section, and switches between the first communication state and the second communication state.

14. The cell culture apparatus according to claim 11,
wherein the opening/closing member includes a support member which maintains a stationary state and a pressing member which is located on a side opposite to the support member across the flexible branching section, and
wherein the opening/closing member moves the pressing member in one direction, and presses and closes the desired branching flow passage so as to switch between the first communication state and the second communication state.

15. The cell culture apparatus according to claim 13,
wherein the support member is located on a lower surface of the flexible branching section, and has a cross shape or a T-shape in a plan view,
wherein the pressing member has a T-shape in a vertical projection plane, and includes a first pressing portion which extends in one direction while facing the support member, and a second pressing portion which extends in a direction perpendicular to the first pressing portion, and
wherein the second pressing portion has a cross-sectional shape which is inclined upward from a connection portion connected to the first pressing portion, which is bent at a predetermined position, and which is inclined downward at a predetermined angle, and is connected to a hinge disposed in the support member in the connection portion so as to pivot in an arc shape around the hinge serving as a fulcrum.
